(19) United States
(12) Patent Application Publication
(10) Pub. No.: US 2026/0063641 A1
(43) Pub. Date: Mar. 5, 2026

(54) COMPOSITIONS AND METHODS FOR LABELING AND DETECTING BINDING PARTNERS

(71) Applicants: UNIVERSITY OF UTAH RESEARCH FOUNDATION, Salt Lake City, UT (US); THE REGENTS OF THE UNIVERSITY OF CALIFORNIA, Oakland, CA (US)

(72) Inventors: Justin G. ENGLISH, Salt Lake City, UT (US); Fleur FERGUSON, La Jolla, CA (US)

(21) Appl. No.: 19/100,557

(22) PCT Filed: Aug. 1, 2023
(86) PCT No.: PCT/US2023/029251

§ 371 Date: Jan. 31, 2025

Related U.S. Application Data
(60) Provisional application No. 63/394,145, filed on Aug. 1, 2022.
(60) Provisional application No. 63/458,612, filed on Apr. 11, 2023.

(51) Int. Cl. G01N 33/58; G01N 33/94

(52) U.S. Cl. CPC G01N 33/581; G01N 33/582; G01N 33/94; G01N 2333/9015

(57) ABSTRACT

Disclosed are compositions comprising a ligandable tag, a linker, and a labeling enzyme, wherein the linker attaches the ligandable tag to the labeling enzyme. Disclosed are nucleic acid sequences capable of encoding one or more of the disclosed protein based compositions. Disclosed are compositions comprising a ligand conjugated to a molecule of interest. Disclosed are systems comprising a labeling composition and a targeting composition, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition. Disclosed are methods of using the disclosed compositions and systems.

### CROSS-REFERENCE TO RELATED APPLICATIONS

This application claims the benefit of U.S. Provisional Patent Application No. 63/394,145, filed Aug. 1, 2022 and U.S. Provisional Patent Application No. 63/458,612, filed Apr. 11, 2023, each of which is incorporated by reference herein in its entirety.

### BACKGROUND

Understanding the interactions between small molecules and proteins can be approached from different perspectives and is essential for advancing fundamental science and drug development. Chemists often use bioactive small molecules, such as natural products or synthetic compounds, as probes to identify therapeutically relevant protein targets. Biochemists and biologists usually begin with a specific protein and seek to identify the endogenous metabolites that bind to it. These interests have led to the development of methodologies that rely heavily on synthetic and analytical chemistry to identify protein-small molecule and protein-metabolite interactions.

One of the successes in using small molecules as affinity reagents is identifying FK506 binding protein by the natural product FK506. This work led to the discovery of new proteins and pathways that explained the mechanism of action of a potent class of immunosuppressant drugs. The increased use of small molecule screening approaches in biology has led to the identification of many bioactive molecules, leading to an increased demand for methods to elucidate protein-small molecule interactions. Today, affinity-based methods are still the most common approach used, and leading methods have leamed to integrate these approaches with modern proteomics to accelerate targeted discovery.

STLAC (stable isotope labeling of amino acids in cell culture) combined with small molecule-affinity chromatography, a quantitative mass spectrometry (MS)-based proteomics strategy (using an ion trap MS), is often used to identify protein-small molecule interactions on a proteome-wide scale. In this method, cells are grown in heavy or light media and subsequently lysed. These heavy and light cell lysates are separately incubated with beads modified with a small molecule of interest. The excess small molecule is added to the light cell lysate, and this soluble small molecule prevents any specific small molecule-protein interactions with the beads. After removing the lysate, bound proteins are eluted from the beads, and the lysates are then combined for subsequent analysis using quantitative proteomics. The samples can be distinguished by mass spectrometry since proteins from each sample have different molecular weights. Therefore, specific protein-small molecule interactions can be identified by the higher concentrations of these heavy proteins versus light proteins.

Significantly, affinity methods are not limited to synthetic compounds or natural products but can also be used with endogenous metabolites. A new group of powerful proteomics methods can be used when small molecules are difficult to modify, or the synthetic skill necessary to make modifications is difficult to access. These strategies rely on detecting differences in the stability between unbound and small molecule-bound proteins to identify the targets of a small molecule.

Drug affinity responsive target stability (DARTS) is one such method. DARTS relies on the fact that proteins are more stable when bound to a metabolite, making them less susceptible to proteolysis. Lysates are compared in the presence and absence of a small molecule. Proteins that bind to the small molecule are protected from proteolysis relative to the control sample, as indicated by mass spectrometry.

Most recently, a proteomics method called stability of proteins from oxidation rates (SPROX) was developed to identify PSMIs in complex cell lysates. Rather than relying on nonspecific proteolysis, which can make downstream mass spectrometry experiments challenging to interpret, SPROX relies on the irreversible oxidation of methionine residues by hydrogen peroxide to report on the thermodynamic stability of a protein's structure during chemical denaturation. Unfortunately, not all proteins are as active in lysates as they are within the context of a cell. Therefore, some relevant protein-small molecule interactions may be missed when using lysates.

Despite the critical role of small molecules in drug discovery and development, there is a lack of comprehensive, network-scale profiling methods that inform on the cellular interactomes of small molecules.

### BRIEF SUMMARY

Disclosed are compositions comprising a ligandable tag, a linker, and a labeling enzyme, wherein the linker attaches the ligandable tag to the labeling enzyme.

Disclosed are nucleic acid constructs comprising a nucleic acid sequence capable of encoding a ligandable tag, a nucleic acid sequence capable of encoding a linker, and a nucleic acid sequence capable of encoding a labeling enzyme.

Disclosed are compositions comprising a ligand conjugated to a molecule of interest.

Disclosed are systems comprising a labeling composition and a targeting composition, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition.

Disclosed are methods of detecting a binding partner of a molecule of interest comprising adding a labeling composition and a target composition to a sample, wherein the labeling composition comprises a ligandable tag conjugated to a labeling enzyme, wherein the target composition comprises a ligand and a molecule of interest, wherein the ligand of the target composition is a ligand for the ligandable tag of the labeling composition; incubating the labeling composition and target composition with the sample under conditions that allow the molecule of interest to bind to a binding partner in the sample, wherein when the molecule of interest binds to a binding partner in the sample, the labeling enzyme labels the binding partner, detecting the presence of the label in the sample, wherein the presence of a label is indicative of a binding partner of a molecule of interest present in the sample.

Disclosed are methods of identifying a binding partner of a molecule of interest comprising adding a labeling composition and a targeting composition to a sample, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition; incubating the labeling composition and targeting composition with the sample under conditions that allow the molecule of interest to bind to a binding partner in the sample, wherein when the molecule of interest binds to a binding partner in the sample, wherein the labeling enzyme labels the binding partner; detecting the presence of the label in the sample, and identifying the labeled binding partner.

The advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1E show the BioTAC system enables rapid, accurate small molecule target-ID. FIG. 1A. Schematic depicting the components of the BioTAC system. FIG. 1B. Example molecule, Cpd1, annotated with key functional groups. FIG. 1C. Immunoblot analysis of BRD4 enrichment following treatment of HEK293 cells transiently transfected with miniTurboFKBP12F36V with the indicated compounds and 100 μM biotin at the 30 min timepoint. Data representative of n=2 biologically independent experiments FIG. 1J). FIG. 1D. Scatterplot displaying relative FC of streptavidin-enriched protein abundance following treatment of HEK293 cells transiently transfected with miniTurboFKBP12F36V with the indicated compounds and 100 μM biotin at the 30 min timepoint. Only proteins with P-value<0.05 in both conditions depicted. Complete datasets are plotted individually in FIG. 6. FIG. 1E. F-test analysis of proteomic data depicted in FIG. 1D, showing significant enrichment of BRD3 and BRD4 in the presence of 1 μM Cpd1, relative to DMSO and (+)-JQ1 off-compete experiments. F-statistic listed top right, X=scaled abundance ratio.

FIGS. 2A-2D show the BioTAC system enables rapid, accurate small molecule interactome-ID. FIG. 2A. Time-course proteomics of streptavidin-enriched biotinylated proteins isolated from HEK293 cells transiently transfected with miniTurboFKBP12^{F36V} and treated with the indicated compounds and 100 μM biotin, demonstrating enrichment and competition of known direct targets (30 min) and complexed proteins (60 min, 4 hrs). Only proteins with P-value<0.05 in both conditions depicted. Complete datasets plotted individually FIG. 6. High-confidence hits are defined as those that are enriched >2-fold in both Cpd1/DMSO and Cpd1/Cpd1+10× (+)-JQ1, where P<0.05, plotted upper-right quadrant. FIG. 2B. Fold-change of all reference BRD2, BRD3, and BRD4 interactors (Lambert et. al.) enriched in the Cpd1 vs. DMSO dataset at the 4 hr time point, showing statistically significant rescue (P<0.0001, 1-way ANOVA). FIG. 2C. Percent enrichment of reference interactors (Lambert et. al.) in the hits identified at the 4 hr timepoint (dotted line), vs. the percent enrichment of known interactors in 4,000 random protein sets of equivalent size from the DMSO control (gray bars), showing significant enrichment by Cpd1 BioTAC (>7 standard deviations away from mean of random chance). FIG. 2D. Gene Ontology analysis, showing significant enrichment of biological processes associated with known BET-protein function in the 4 hr Cpd1 BioTAC experiment hits.

FIGS. 3A-3C shows the BioTAC system enables detection of non-degrader molecular glue interactions. FIG. 3A. Schematic depicting how the BioTAC system can detect molecular glue interactions. FIG. 3B. Chemical structure of Trametinib recruiting bifunctional molecule JWJ-01-280-1/Cpd2. FIG. 3C. Immunoblot analysis of MEK1 and KSR1 following treatment of HEK293 cells transiently transfected with miniTurboFKBP12^{F36V} with the indicated compounds/growth factors and 100 μM biotin at the 4 h timepoint, and streptavidin-based enrichment, showing successful enrichment and competition with trametinib.

FIGS. 4A-4J shows optimization of (+)-JQ1 orthoAP1867 bifunctional molecules. FIG. 4A.-FIG. 4C. Chemical structure of all tested (+)-JQ1-linker-ortho-AP1867 analogues. FIG. 4D. FKBP12^{F36V} cellular target engagement assays for compounds shown FIG. 4A-FIG. 4C, data plotted as mean±S.D. of n=3 technical replicates. FIG. 4D. FMF-06-147-1, FIG. 4E. JWJ-01-255, FIG. 4F. JWJ-01-236, demonstrating competitive displacement of dTAG-13. FIG. 4G. Immunoblot analysis of BRD4 enrichment following treatment of HEK293 cells transiently transfected with miniTurboFKBP12^{F36V} and treated with FMF-06-147-1 and 100 μM biotin at the indicated timepoint. FIG. 4H. Immunoblot analysis of BRD4 enrichment following treatment of HEK293 cells transiently transfected with miniTurboFKBP12^{F36V} and treated with the indicated compounds and 100 μM biotin at the 60 min timepoint. FIG. 4I. Immunoblot analysis of BRD4 enrichment following treatment of HEK293 cells transiently transfected with miniTurboFKBP12^{F36V} and treated with FMF-06-147-1 and 100 μM biotin at the 30 min timepoint. FIG. 4J. Replicate 2 of immunoblot analysis of BRD4 enrichment following treatment of HEK293 cells transiently transfected with miniTurboFKBP12^{F36V} and treated with FMF-06-147-1 and 100 μM biotin at the 30 min timepoint. FIG. 4G, FIG. 4I. Data representative of n=3 biologically independent experiments. FIG. 4J. Data representative of n=2 biologically independent experiments. S.D. Standard deviation

FIGS. 5A-5C show a comparison of the BioTAC system to photoaffinity labeling and μMap. FIG. 5A-FIG. 5B. Replotted reference data from Trowbridge et. al. showing photoaffinity labeling and μMap results for (+)-JQ1. FIG. 5C. Proteomics analysis of biotinylated proteins enriched by streptavidin pulldown from HEK293 cells transiently transfected with miniTurbo-FKBP12^{F36V} and treated with 100 μM Biotin, 1 μM of Cpd1±10 μM (+)-JQ1 for 30 mins.

FIGS. 6A-6C show Volcano plots of proteomic experiments. Volcano plots showing all data points from mass-spectrometry based proteomic analysis of proteins enriched by streptavidin pulldown from HEK293 cells transiently transfected with miniTurbo-FKBP12^{F36V} and treated with 100 μM Biotin, DMSO or 1 μM of Cpd1±10 μM (+)-JQ1 for FIG. 6A. 30 mins. FIG. 6B. 60 mins. FIG. 6C. 4 hrs. Points corresponding to BRD3 and BRD4 are highlighted blue.

FIGS. 7A-7C show optimization of MEK1 labeling by trametinib bifunctional molecules. FIG. 7A. FKBP12^{F36V} cellular target engagement assays for JWJ-01-280, data plotted as mean±S.D. of n=3 technical replicates. FIG. 7B. Immunoblot analysis of MEK1/2 enrichment following treatment of HEK293 cells transiently transfected with miniTurboFKBP12^{F36V} with JWJ-01-280-1 and 100 μM biotin at the indicated timepoint. FIG. 7C. Immunoblot analysis of MEK1/2 and KSR1 enrichment following treatment of HEK293 cells transiently transfected with miniTurboFKBP12^{F36V} with the indicated concentration of JWJ-01-280-1 and 100 μM biotin for 4 h. Data representative of n=3 biologically independent experiments. S.D. Standard deviation.

FIGS. 8A-8F show FIG. 8A. Uncut blot of main text FIG. 1C and FIG. 4J (indicated). FIG. 8B. Uncut blot of main text FIG. 3C and FIG. 7C (indicated). FIG. 8C. Uncut blot of FIG. 4G. FIG. 8D. Uncut blot of FIG. 4H. FIG. 8E. Uncut blot of FIG. 4I. F. Uncut blot of FIG. 7B.

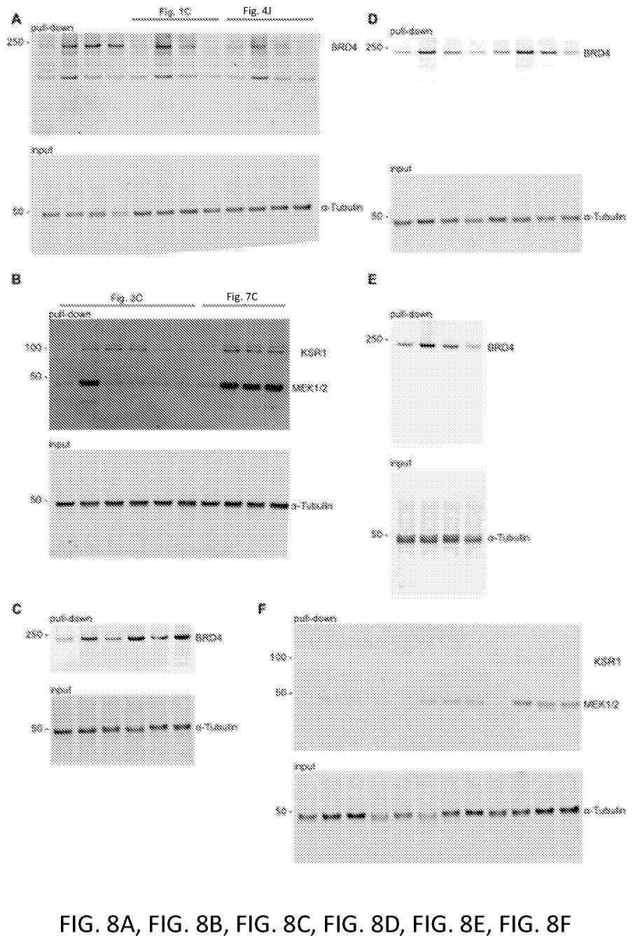

FIGS. 9A-9C show components of the BIOTAC system. FIG. 9A) Construct design for N- or C-terminally tagged miniTurboID. FIG. 9B) Small molecule design for example compound JQ1. FIG. 9C) Cellular FKB12^{F36V} target engagement assay demonstrating cell permeability of the compound.

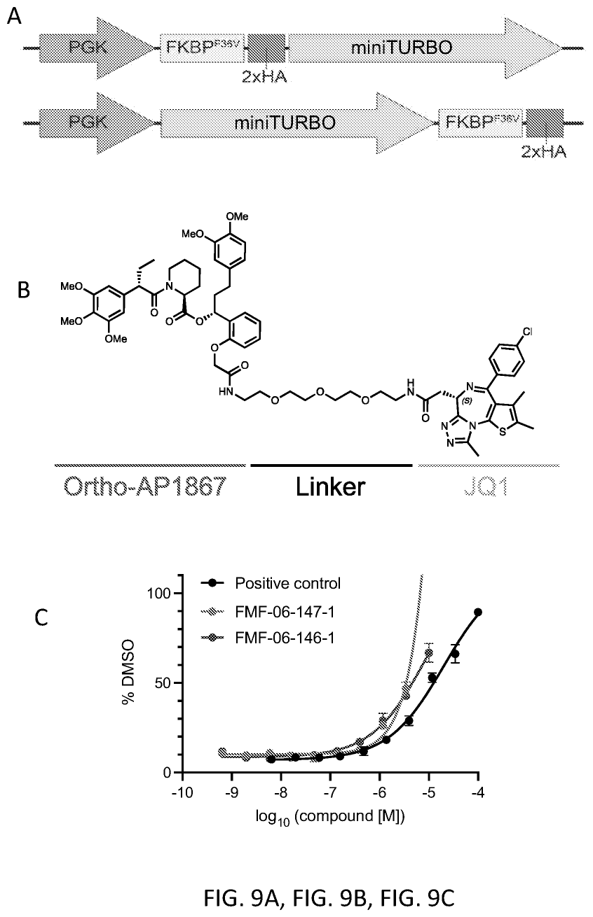

FIG. 10 shows a pcDNA3.1 vector construct comprising BIOTAC (6662 bp).

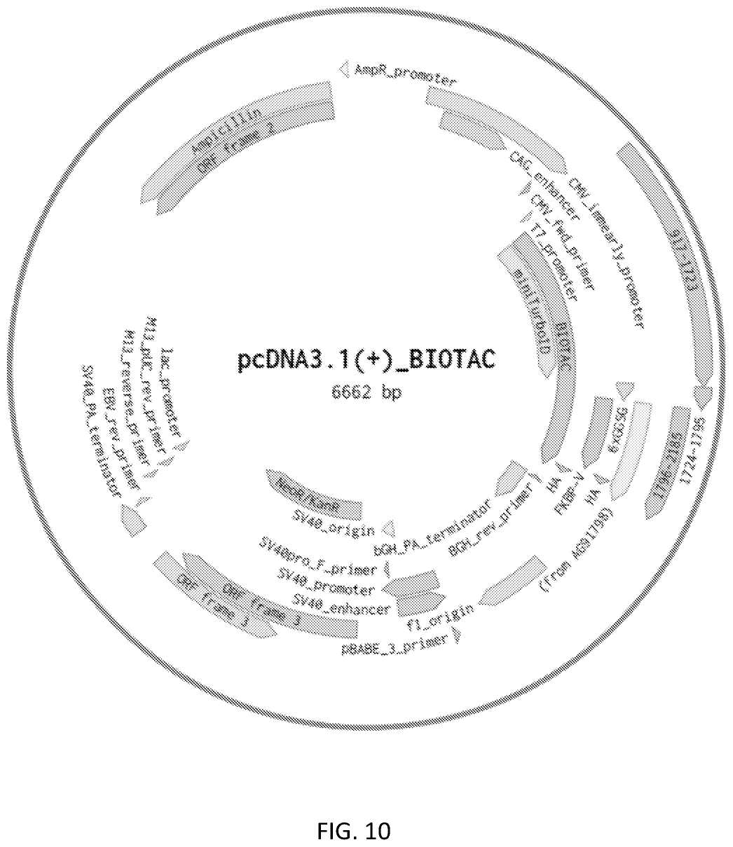

FIGS. 11A and 11B show an example that the BioTAC compound FMF-06-147 identifies known BET-containing complexes. FIG. 11A) Violin plot of log 2FC enrichment of all proteins identified in our (+)-JQ1 BioTAC experiment (FIG. 9) that were also identified in Lambert et al. N=3, statistical analysis One-Way ANOVA with multiple comparisons. ****=p<0.0001. FIG. 11B) Heatmap showing mass-spectrometry based quantification of enriched proteins following treatment with 1 μM (+)-JQ1-BioTAC in the presence of DMSO, 10 μM (+)-JQ1 or 100 μM (+)-JQ1. Proteins are grouped based on complex ID. Significant, FC>3. P<0.05. Trending, proteins identified that displayed the expected enrichment and competition profiles but did not meet significance cutoffs. Random sample, random selection of detected proteins without any enrichment criteria applied.

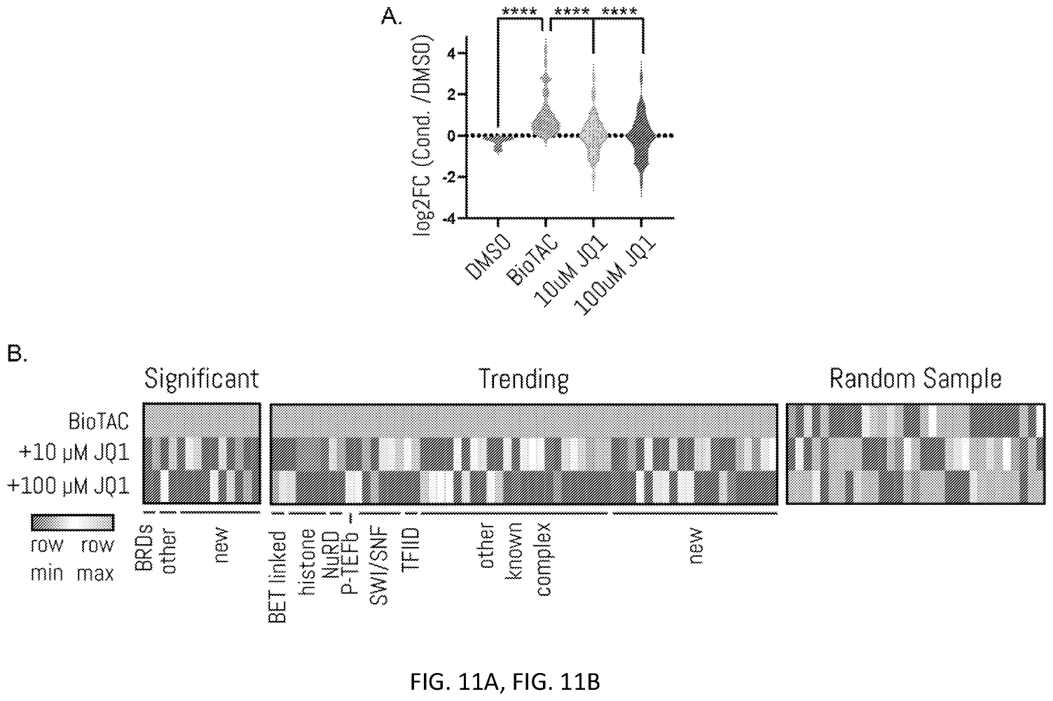

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a ligandable tag” includes a plurality of such ligandable tags, reference to “the ligand” is a reference to one or more ligands and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

### 1. Ligandable Tag and Labeling Enzyme (Labeling Composition)

i. Protein Based Compositions

Disclosed are compositions comprising a ligandable tag, a linker, and a labeling enzyme, wherein the linker attaches the ligandable tag to the labeling enzyme.

a. Ligandable Tag

The term “ligandable tag” as used herein, shall mean any genetically encodable ligand binding domain. For example, a “ligandable tag” can be a domain that can bind to one or more ligands selectively. In some aspects, the ligandable tag selectively recognizes a ligand that does not bind to any endogenous proteins in the cell.

In some aspects, the ligandable tag is a peptide, nucleic acid, or anything that can be genetically encoded. In some aspects, the ligandable tag is FKBP12^{F36V}, HaloTAG, SNAP-tag, CLIP-TAG, split inteins (e.g. M. tuberculosis RecA intein donor, SpyCatcher, or LigandLink. In some aspects, non-limiting examples of ligands for the ligandable tags are provided in Table 1.

TABLE 1
Examples of ligandable tags and their ligand bind partners

| Ligandable Tag | Ligand |
|---|---|
| FKBP12^{F36V} | Ortho-AP1867 |
| Halo TAG | Haloalkanes (e.g. JF549) |
| SNAP-tag | O⁶-benzylguanine |
| | O⁶-benzylguanine derivatives |
| | benzyl-2-chloro-6-aminopyrimidines |
| CLIP-TAG | benzylcytosine derivatives |
| M. tuberculosis RecA intein donor | M. tuberculosis RecA intein acceptor |
| SpyCatcher | SpyTag |
| LigandLink or TMP-Tag | trimethoprim derivatives |

In some aspects, the ligandable tag can be linked (e.g. covalently) by a linker to the labeling enzyme.

In some aspects, a ligandable tag is capable of binding with high affinity to its ligand.

b. Labeling Enzyme

The term “labeling enzyme” as used herein refers to any enzyme that is capable of labeling a molecule of interest for subsequent purification or biochemical analysis. In some aspects, a labeling enzyme is any enzyme that can covalently attach a molecule to a molecule of interest (e.g. protein) to enable subsequent downstream purification and/or analysis. In some aspects, the labeling enzyme can be any genetically encodeable enzyme that can add or attach a label to a molecule of interest. In some aspects, the molecule of interest that can be labeled by the disclosed labeling enzymes can be, but is not limited to, a peptide, a protein, a compound (e.g. biotin or a synthetic molecule, or a molecule containing a click-chemistry/biorthogonal chemistry handle), fatty acid, sterol, or nucleic acid. In some aspects, the label attached to a molecule of interest via the labeling enzyme can be, but is not limited to, biotin, methyl group, farnesyl group, and azide-bearing lipoic acid derivative.

In some aspects, the labeling enzyme is an enzyme which can label a protein for biochemical analysis. For example, in some aspects, the labeling enzyme can be a biotin ligase, or a peroxidase (e.g. ascorbic acid peroxidase; APEX). In some aspects, a labeling enzyme is an engineered enzyme for proximity labeling applications. In some aspects, a protein ligase is a protein having ligase activity. In some aspects, the labeling enzyme can be a biotin ligase, methylase, Formlyglycine-generating enzyme, sortase, transglutaminase, famesyltransferase, APEX, or lipoic acid ligase. In some aspects, the protein ligase can be a biotin ligase and the biotin ligase can be miniTurboID, TurboID, AirID, splitTurboID, or BirA.

In some aspects, the labeling enzyme can be encoded by the same construct that encodes the ligandable tag.

In some aspects, the labeling enzyme can be linked by a linker to the ligandable tag. In some aspects, the link between the labeling enzyme and the ligandable tag is covalent.

c. Linker

Disclosed are linkers that can attach a ligandable tag to a labeling enzyme.

In some aspects, the linker can be a flexible linker. In some aspects, a flexible linker allows the ligandable tag and/or the labeling enzyme to move close to the ligand and/or molecule of interest, respectively. In some aspects, the linker is chemically synthesizable and conjugatable.

In some aspects, the linker is not proteolytic, and thus cannot be cleaved by an enzyme or other protein.

In some aspects, the linker can vary in length. In some aspects the linker can be 1-500 amino acids in length. In some aspects, a longer linker can be used to allow the labeling enzyme to label a molecule of interest that is further away. In some aspects, a longer linker can be a linker greater than 15, 20, 25, 30, 35, 40, 45, 50 amino acids.

In some aspects, the linkers can be protein based linkers. Thus, in some aspects, the linkers are genetically encodable.

In some aspects, the linkers can be standard, known linkers, short strings of random amino acids; flexible linkers such as (G)n/(GX)n/(GGX)n/(GGGX)n; semiflexible linkers such as (GS)n, (G)n/(S)n combinations, (D/E)n, or (PAS)n shuffled sequences; structured linkers such as (EAAAK)n, (EAQA)n, or (MALEK)n; or any other linkers engineered to junction two functional protein domains with flexible, defined, or disordered structural compositions, short strings of random amino acids; flexible linkers such as (G)n/(GX)n/(GGX)n/(GGGX)n; semiflexible linkers such as (GS)n, (G)n/(S)n combinations, (D/E)n, or (PAS)n shuffled sequences; structured linkers such as (EAAAK)n, (EAQA)n, or (MALEK)n; or any other linkers engineered to junction two functional protein domains with flexible, defined, or disordered structural compositions. In some aspects, the linker is (GGSGS)₆.

In some aspects, the linker is present at the N-terminal end of the ligandable tag. In some aspects, the linker attaches the C-terminal end of the labeling enzyme with the N-terminal end of the ligandable tag. In some aspects, the linker attaches the N-terminal end of the labeling enzyme with the N-terminal end of the ligandable tag.

In some aspects, the linker is present at the C-terminal end of the ligandable tag. In some aspects, the linker attaches the C-terminal end of the labeling enzyme with the C-terminal end of the ligandable tag. In some aspects, the linker attaches the N-terminal end of the labeling enzyme with the C-terminal end of the ligandable tag.

d. Additional Elements

In some aspects, the disclosed compositions comprising a ligandable tag, a linker, and a labeling enzyme can further comprise a detection moiety or a purification moiety. In some aspects, the detection moiety or purification moiety can be used to detect or purify the composition after it has been produced. For example, if the disclosed compositions are produced by transfecting cells with one or more of the disclosed constructs that encode one or more of the disclosed compositions, then the detection moiety can be used to detect the presence of the composition in the cells and/or the purification moiety can be used to purify the composition away from the other cellular material.

In some aspects, the detection moiety or purification moiety is a fluorescent molecule, biotin, strep tag, c-myc, HA, GST, histidines, or Flag tag.

In some aspects, the disclosed compositions comprising a ligandable tag, a linker, and a labeling enzyme can further comprise a subcellular localization moiety or inducible targeting moiety. In some aspects, the localization or targeting moiety can be used to induce localization of the composition after it has been produced. For example, if the disclosed compositions are produced by transfecting cells with one or more of the disclosed constructs that encode one or more of the disclosed compositions, then the localization or targeting moiety can be used to concentrate the composition within a subcellular domain of the cell, transport the composition into a subcellular compartment, or co-localize the composition with another endogenous or exogenous element within or on the cell.

In some aspects, the localization or targeting moiety is a cellular address tag, MitoTag, nuclear localization sequence, nuclear export sequence, secretion tag, nanobody, intein, dimerizing agent, or sorting tag.

e. Example

In some aspects, a composition comprising a ligandable tag, a linker, and a labeling enzyme comprises the amino acid sequence:

(SEQ ID NO: 1)
MARDPPVATMIPLLNAKQILGQLDGGSVAVLPVVDSTNQYLLDRIGELKS
GDACIAEYQQAGRGSRGRKWFSPFGANLYLSMFWRLKRGPAAIGLGPVIG
IVMAEALRKLGADKVRVKWPNDLYLQDRKLAGILVELAGITGDAAQIVIG
AGINVAMRRVEESVVNQGWITLQEAGINLDRNTLAAMLIRELRAALELFE
QEGLAPYLSRWEKLDNFINRPVKLIIGDKEIFGISRGIDKQGALLLEQDG
VIKPWMGGEISLRSAEKASGGSGGGSGGGSGGGSGGGSGGGSGGVQVETI
SPGDGRTFPKRGQTCVVHYTGMLEDGKKVDSSRDRNKPFKFMLGKQEVIR
GWEEGVAQMSVGQRAKLTISPDYAYGATGHPGIIPPHATLVFDVELLKLE
GGYPYDVPDYAGYPYDVPDYAG*.
The all caps sequence is miniTurboID (i.e.
labeling enzyme). The bold all caps is (GGSG)₆
(SEQ ID NO: 2) linker. The italicized all caps is
FKBP12^{F36V}(i.e. ligandable tag).

ii. Nucleic Acid Based Compositions

Disclosed are nucleic acid sequences capable of encoding one or more of the disclosed protein based compositions. Disclosed are constructs comprising one or more of the disclosed nucleic acid sequences. Disclosed are nucleic acid constructs comprising a nucleic acid sequence capable of encoding a ligandable tag, a nucleic acid sequence capable of encoding a linker, and a nucleic acid sequence capable of encoding a labeling enzyme.

In some aspects, the nucleic acid sequence can further comprise a nucleic acid sequence capable of encoding a detection moiety or purification moiety. In some aspects, the detection moiety or purification moiety is a fluorescent molecule, biotin, c-myc, HA, GST, histidines. In some aspects, the detection moiety or purification moiety is a fluorescent molecule, biotin, c-myc, HA, GST, histidines.

Disclosed are constructs comprising a nucleic acid sequence that encodes a ligandable tag, a linker, and/or a labeling enzyme. In some aspects, all of the ligandable tag, linker, and labeling enzyme are encoded by the same construct. In some aspects, each of the ligandable tag, linker, and labeling enzyme are encoded by separate nucleic acid sequences.

a. Vectors

In some aspects, a construct comprising a nucleic acid sequence that encodes a ligandable tag, a linker, and/or a labeling enzyme can be a vector.

Disclosed are vectors comprising one or more of the nucleic acid sequences disclosed herein.

In some aspects, the vector can be a plasmid comprising the sequence

(SEQ ID NO: 3)
GACGGATCGGGAGATCTCCCGATCCCCTATGGTGCACTCTCAGTACAATCTGCTCTG
ATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATG
AAGAATCTGCTTAGGGTTAGGCGTTTTGCGCTGCTTCGCGATGTACGGGCCAGATAT
ACGCGTTGACATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAAC
TGCCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGACTT
TCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCC
AAAATGTCGTAACAACTCCGCCCCATTGACGCAAATGGGCGGTAGGCGTGTACGGT
GGGAGGTCTATATAAGCAGAGCTCTCTGGCTAACTAGAGAACCCACTGCTTACTGG
CTTATCGAAATTAATACGACTCACTATAGGGAGACCCAAGCTGGCTAGCGTTTAAA
CTTAAGCTTatggctcgggatccaccggtcgccaccatgatcccgctgctgaacgctaaacagattctgggacagctggacggcg
ggagcgtggcagtcctgcctgtggtcgactccaccaatcagtacctgctggatcgaatcggcgagctgaagagtggggatgcttgcattgc
agaatatcagcaggcagggagaggaagcagagggaggaaatggttctctccttttggagctaacctgtacctgagtatgttttggcgcctg
aagcggggaccagcagcaatcggcctgggcccggtcatcggaattgtcatggcagaagcgctgcgaaagctgggagcagacaaggtg
cgagtcaaatggcccaatgacctgtatctgcaggatagaaagctggcaggcatcctggtggagctggccggaataacaggcgatgctgc
acagatcgtcattggcgccgggattaacgtggctatgaggcgcgtggaggaaagcgtggtcaatcagggctggatcacactgcaggaag
cagggattaacctggacaggaatactctggccgctatgctgatccgagagctgcgggcagccctggaactgttcgagcaggaaggcctg
gctccatatctgtcacggtgggagaagctggataacttcatcaatagacccgtgaagctgatcattggggacaaagagattttcgggattag
ccgggggattgataaacagggagccctgctgctggaacaggacggagttatcaaaccctggatgggcggagaaatcagtctgcggtctg
ccgaaaaggctagcGGTGGTTCTGGTGGCGGCAGTGGGGGGGGGTCCGGTGGCGGAT
CAGGTGGTGGAAGTGGCGGTGGCTCAGGTggagtgcaggtggaaaccatctccccaggagacgggc
gcaccttccccaagcgcggccagacctgcgtggtgcactacaccgggatgcttgaagatggaaagaaagttgattcctcccggga
cagaaacaagccctttaagtttatgctaggcaagcaggaggtgatccgaggctgggaagaaggggttgcccagatgagtgtggg
tcagagagccaaactgactatatctccagattatgcctatggtgccactgggcacccaggcatcatcccaccacatgccactctcgt
cttcgatgtggagcttctaaaactggaaggcggctacccctacgacgtgcccgactacgccggctatccgtatgatgtcccgacta
tgcaggctaaGAATTCTGCAGATATCCAGCACAGTGGCGGCCGCTCGAGTCTAGAGGGCCCG
TTTAAACCCGCTGATCAGCCTCGACTGTGCCTTCTAGTTGCCAGCCATCTGTTGTTTGCCC
CTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGGATGCGGTG
GGCTCTATGGCTTCTGAGGCGGAAAGAACCAGCTGGGGCTCTAGGGGGTATCCCCACGC
GCCCTGTAGCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTCCGATTTAGTG
CTTTACGGCACCTCGACCCCAAAAAACTTGATTAGGGTGATGGTTCACGTAGTGGGCCAT
CGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGA
TTTTGCCGATTTCGGCCTATTGGTTAAAAAATGAGCTGATTTAACAAAAATTTAACGCGAAT
TAATTCTGTGGAATGTGTGTCAGTTAGGGTGTGGAAAGTCCCCAGGCTCCCCAGCAGGCA
GAAGTATGCAAAGCATGCATCTCAATTAGTCAGCAACCAGGTGTGGAAAGTCCCCAGGCT
CCCCAGCAGGCAGAAGTATGCAAAGCATGCATCTCAATTAGTCAGCAACCATAGTCCCGC
CCCTAACTCCGCCCATCCCGCCCCTAACTCCGCCCAGTTCCGCCCATTCTCCGCCCCATG
GCTGACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAGCTTG
TATATCCATTTTCGGATCTGATCAAGAGACAGGATGAGGATCGTTTCGCATGATTGAACAA
GATGGATTGCACGCAGGTTCTCCGGCCGCTTGGGTGGAGAGGCTATTCGGCTATGACTG
GGCACAACAGACAATCGGCTGCTCTGATGCCGCCGTGTTCCGGCTGTCAGCGCAGGGGC
GCCCGGTTCTTTTTGTCAAGACCGACCTGTCCGGTGCCCTGAATGAACTGCAGGACGAG
GCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGG
CTGCATACGCTTGATCCGGCTACCTGCCCATTCGACCACCAAGCGAAACATCGCATCGAG
CGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGCGCATGCCCGACGGC
GAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTTCC
TCGTGCTTTACGGTATCGCCGCTCCCGATTCGCAGCGCATCGCCTTCTATCGCCTTCTTG
ACGAGTTCTTCTGAGCGGGACTCTGGGGTTCGAAATGACCGACCAAGCGACGCCCAACC
TGCCATCACGAGATTTCGATTCCACCGCCGCCTTCTATGAAAGGTTGGGCTTCGGAATCG
TTTTCCGGGACGCCGGCTGGATGATCCTCCAGCGCGGGGATCTCATGCTGGAGTTCTTC
GCCCACCCCAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATCAATGT
ATCTTATCATGTCTGTATACCGTCGACCTCTAGCTAGAGCTTGGCGTAATCATGGTCATAG
CTGTTTCCTGTGTGAAATTGTTATCCGCTCACAATTCCACACAACATACGAGCCGGAAGCA
TAAAGTGTAAAGCCTGGGGTGCCTAATGAGTGAGCTAACTCACATTAATTGCGTTGCGCTC
ACTGCCCGCTTTCCAGTCGGGAAACCTGTCGTGCCAGCTGCATTAATGAATCGGCCAACG
CGCGGGGAGAGGCGGTTTGCGTATTGGGCGCTCTTCCGCTTCCTCGCTCACTGACTCGC
TGCGCTCGGTCGTTCGGCTGCGGCGAGCGGTATCAGCTCACTCAAAGGCGGTAATACGG
TTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAGTTGGTAGCTC
TTGATCCGGCAAACAAACCACCGCTGGTAGCGGTTTTTTTGTTTGCAAGCAGCAGATTACG
CGCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCTA
GATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCAT
CCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTATGGCTT
CATTCAGCTCCGGTTCCCAACGATCAAGGCGAGTTACATGATCCCCCATGTTGTGCAAAA
AAGCGGTTAGCTCCTTCGGTCCTCCGATCGTTGTCAGAAGTAAGTTGGCCGCAGTGTTAT
CACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAG
TTGCTCTTGCCCGGCGTCAATACGGGATAATACCGCGCCACATAGCAGAACTTTAAAAGT
GCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCC
GCGCACATTTCCCCGAAAAGTGCCACCTGACGTC.
All caps represent a vector backbone and promoter sequence. Lower case letters represent
miniTurboID. Bold all caps represent the (GGSG)₆ (SEQ ID NO: 2) linker. Bold lower case
represents FKBP12^{F36V}. Bold lower case and underlined represents 2xHA tag. Italicized all
caps represents bGH terminator sequence and vector backbone.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vectors are used to deliver the disclosed nucleic acid sequences to cells to ultimately result in expression of the proteins encoded by the nucleic acid sequences. These proteins can then be used in the methods disclosed herein.

In some aspects, the vector can be a viral vector. For example, the viral vector can be an adeno-associated viral vector (AAV). In some aspects, the AAV can be AAV9. In some aspects, the vector can be a non-viral vector, such as a DNA based vector.

In some aspects, the vector is AAV9. In some aspects, the disclosed vectors are considered recombinant vectors. A recombinant AAV can lack two essential genes for viral integration and replication but remains primarily episomal and can persist in non-dividing cells for long periods of time.

### (A) Viral and Non-Viral Vectors

There are a number of compositions and methods which can be used to deliver the disclosed nucleic acids to cells in order to produce the proteins encoded by the nucleic acid sequences. These methods and compositions can largely be broken down into two classes: viral based delivery systems and non-viral based delivery systems. For example, the nucleic acids can be delivered to cells through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence encoding a ligandable tag, a linker and a labeling enzyme.

The “control elements” present in an expression vector are those non-translated regions of the vector—enhancers, promoters e.g. a human pro-B-type natriuretic protein (hBNP) promoter), 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as PGK, the hybrid lacZ promoter of the pBLUESCRIPT phagemid (Stratagene, La Jolla, Calif) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I.M., Retroviral vectors for gene transfer. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene, which is not native to the AAV or B19 parvovirus. Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation.

### 2. Ligand Conjugated to Molecule of Interest (Targeting Composition)

Disclosed are compositions comprising a ligand conjugated to a molecule of interest. In some aspects, the compositions comprising a ligand conjugated to a molecule of interest are not fully protein based, meaning at least one element of the compositions is chemical, or compound, based. Thus, in some aspects, the compositions comprising a ligand conjugated to a molecule of interest are not entirely genetically encodeable.

a. Ligand

In some aspects, a ligand is a molecule that is capable of binding to a ligandable tag. Non-limiting examples of ligands and a corresponding ligandable tag are provided in Table 1.

In some aspects, the ligand binds to a ligandable tag such as, but not limited to, FKBP12^{F36V}, HaloTAG, SNAP-tag, CLIP-TAG, split inteins (e.g. M. tuberculosis RecA intein donor), SpyCatcher, or LigandLink. Thus, in some aspects, the ligand can be, Ortho-API867, Haloalkanes (e.g. JF549), 06-benzylguanine/06-benzylguanine derivatives/benzyl-2-chloro-6-aminopyrimidines, benzylcytosine derivatives, M. tuberculosis RecA intein acceptor, SpyTag, or trimethoprim derivatives, respectively. In some aspects, the BromoTag system can be used.

b. Molecule of Interest

In some aspects, a molecule of interest can be a peptide, protein, nucleic acid sequence, small molecule, or compound. Specifically, in some aspects, a molecule of interest can be an antibody, an enzyme, DNA, or RNA. In some aspects, the molecule of interest can be used as bait to determine what its binding partner (i.e. target of interest) can be. In some aspects, the target of interest is unknown and therefore the molecule of interest can be used to identify the unknown target of interest. In some aspects, the target of interest is known and therefore a specific molecule of interest can be used in order to confirm binding between the molecule of interest and the target of interest. In some aspects, the molecule of interest binds to the target of interest and the target interest can have a set of molecular interactions (e.g. interactome). Thus, in some aspect, the molecule of interest can be used to determine its direct binding partner (e.g. target of interest) or indirect interactions (e.g. interactome).

In some aspects, the molecule of interest is ribociclib, (+)-S-JQ1, or trametinib.

In some aspects, the molecule of interest can be a drug candidate or drug-of-interest.

c. Linkers

In some aspects, the compositions can comprise a linker between the ligand and molecule of interest. In some aspects, the linker can be a chemical linker. In some aspects, the linker is not protein based. In some aspects, the linker is synthesizable. In some aspects, the linker is conjugatable.

In some aspects, the linker can be, but is not limited to, PEGs, hydrocarbons, mixed aliphatic/aromatic ring structures.

In some aspects, the linker can be based on one or more of the following structures: —CH₂—CH₂—O—CH₂—CH₂—; —CH₂—CH₂—(CH₂—O—CH₂)—CH₂—CH₂; —CH₂—(CH₂—O—CH₂)₂—CH₂—; —(CH₂)₃—; —(CH₂)₅—.

d. Examples

Disclosed are examples of compositions comprising a ligand conjugated to a molecule of interest.

In some aspects, the composition can have the following structure:

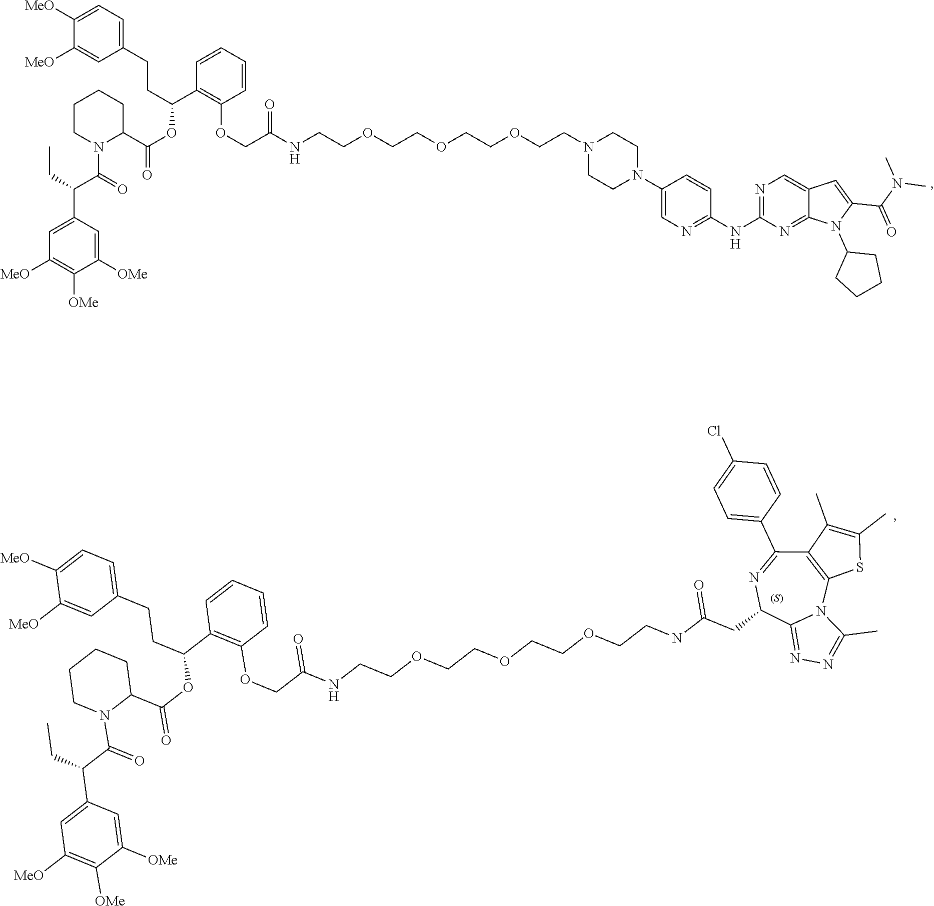

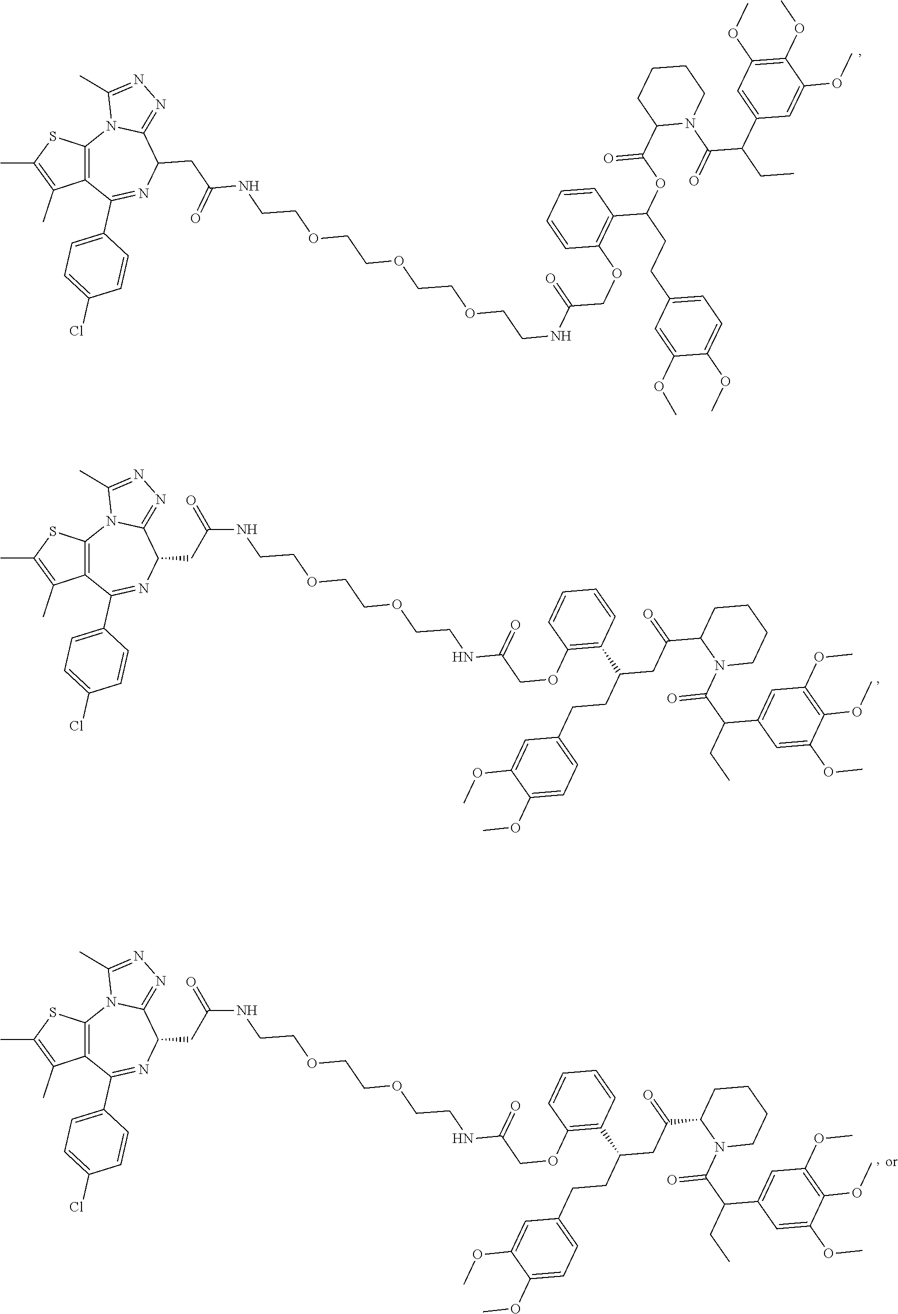

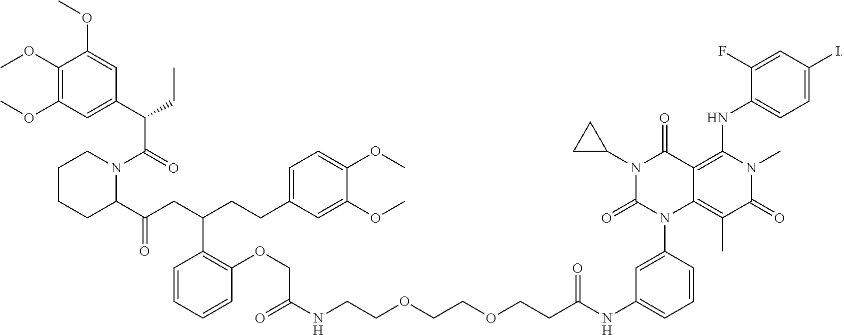 [SMILES: CC[C@H](C(=O)N1CCCCC1C(=O)CC(CCc1ccc(OC)c(OC)c1)c1ccccc1OCC(=O)NCCOCCOCCC(=O)Nc1cccc(-n2c(=O)n(C3CC3)c(=O)c3c(Nc4ccc(I)cc4F)n(C)c(=O)c(C)c32)c1)c1cc(OC)c(OC)c(OC)c1]

ii. Complexes

Disclosed are complexes comprising a first composition comprising a ligandable tag, a linker, and a labeling enzyme, wherein the linker attaches the ligandable tag to the labeling enzyme, bound to or otherwise interacting with a second composition comprising a ligand conjugated to a molecule of interest, wherein the complex is bound via an interaction between the ligandable tag and the ligand.

The disclosed complexes can comprise a combination of the compositions described throughout.

In some aspects, the disclosed complexes can include a labeling composition and a targeting composition, wherein wherein the labeling composition comprises a ligandable tag, a linker, and a labeling enzyme, wherein the linker attaches the ligandable tag to the labeling enzyme and the targeting composition comprises a ligand conjugated to a molecule of interest.

In some aspects, the complexes can comprise the target of interest and/or the target of interest's interactome.

### C. Systems

Disclosed are systems comprising a labeling composition and a targeting composition, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition.

In some aspects, the disclosed systems comprise both the labeling composition and targeting composition bound to each other in order to act a single component able to further bind to a target of interest and optionally label the target of interest.

In some aspects of the disclosed systems, the components can be any of those as described throughout. For example, the ligandable tag, linker and labeling enzyme can be any of those described herein.

### D. Methods

Disclosed are methods of using the one or more of the compositions disclosed throughout.

### 1. Methods of Detecting

Disclosed are methods of detecting a binding partner of a molecule of interest comprising adding a labeling composition and a target composition to a sample, wherein the labeling composition comprises a ligandable tag conjugated to a labeling enzyme, wherein the target composition comprises a ligand and a molecule of interest, wherein the ligand of the target composition is a ligand for the ligandable tag of the labeling composition; incubating the labeling composition and target composition with the sample under conditions that allow the molecule of interest to bind to a binding partner in the sample, wherein when the molecule of interest binds to a binding partner in the sample, the labeling enzyme labels the binding partner, detecting the presence of the label in the sample, wherein the presence of a label is indicative of a binding partner of a molecule of interest present in the sample. In some aspects, the binding partner can be isolated and further characterized. In some aspects, the binding partner can be a target of interest.

In some aspects, the disclosed methods can be performed in vitro or in vivo. Thus, in some aspects, the sample is a cell culture (in vitro) or an animal (in vivo).

In some aspects, adding a labeling composition and a target composition to a sample can be performed simultaneously or consecutively.

In some aspects, incubating the labeling composition and target composition with the sample can occur for at least 1, 2, 3, 4, 5, 6, 7, 8, 9, 10, 12, 14, 16, 18, 20, 22, or 24 hours. In some aspects, the incubating can occur while the sample is rocking or mixing.

In some aspects, detecting the presence of the label in the sample can vary based on what labeling system is used. For example, if the label is biotin then the biotin labeled binding partner can be isolated with streptavidin and then detected using immunoassays and/or imaging.

In some aspects, the sample comprises the binding partner (e.g. a target of interest). In some aspects, the sample is a cell culture, bodily fluid (e.g. blood, plasma, urine, saliva, etc.), or tissue (e.g. brain, muscle, or heart), or whole organism (e.g. mouse, rat, zebrafish, fly, etc).

In some aspects, the labeling enzyme is any enzyme that can label a binding partner (e.g. target of interest), such as a protein, for biochemical analysis. In some aspects, the labeling enzyme is a protein with ligase activity. In some aspects, the protein with ligase activity is a biotin ligase. Formlyglycine, sortase, transglutaminase, farnesyltransferase, or lipoic acid ligase. In some aspects, a biotin ligase can be, but is not limited to, miniTurboID, TurboID, or BirA.

In some aspects, the ligandable tag in the labeling composition binds to its ligand in the targeting composition bringing together the labeling composition and the targeting composition. In some aspects, the ligandable tag is FKBP^{F36V}, Halo TAG, SNAP-tag, CLIP-TAG, split inteins (e.g. M. tuberculosis RecA intein donor), SpyCatcher, or LigandLink. Thus, in some aspects, the ligand (in the target composition) can be, Ortho-AP1867, Haloalkanes (e.g. JF549), 06-benzylguanine/06-benzylguanine derivatives/benzyl-2-chloro-6-aminopyrimidines, benzylcytosine derivatives, M. tuberculosis RecA intein acceptor, SpyTag, or trimethoprim derivatives, respectively.

In some aspects, the targeting composition comprises a linker between the ligand and molecule of interest. In some aspects, the linker is a chemical linker.

In some aspects, the labeling composition comprises a linker between the ligandable tag and labeling enzyme. In some aspects, the linker is an amino acid based linker.

In some aspects of the disclosed method, the ligand of the targeting composition binds to the ligandable tag of the labeling composition. Meanwhile, the molecule of interest on the targeting composition binds to a binding partner (e.g. target of interest) in the sample. Due to the binding of the labeling composition and the target composition, the labeling enzyme on the labeling composition can be placed into close enough proximity to the binding partner in the sample to result in the labeling enzyme attaching a label to the binding partner. In some aspects, any other molecules also bound to the binding partner can also be labeled by the labeling enzyme. In some aspects, this allows for detection of the binding partner and other molecules that interact with the binding partner. Thus, in some aspects, the methods disclosed herein can be used to study an interactome of a binding partner.

In some aspects, detecting the labeled binding partners allows for screening for “off target” binding of the binding partners. For example, the methods disclosed herein can allow for detection of additional binding partners for a molecule of interest.

### 2. Methods of Identifying

Not only can the disclosed compositions be used to detect a binding partner of a molecule of interest present in a target composition, the disclosed compositions can also be used to identify the binding partner (e.g. target of interest). Thus, disclosed are methods of identifying a binding partner of a molecule of interest comprising adding a labeling composition and a targeting composition to a sample, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition; incubating the labeling composition and targeting composition with the sample under conditions that allow the molecule of interest to bind to a binding partner in the sample, wherein when the molecule of interest binds to a binding partner in the sample, wherein the labeling enzyme labels the binding partner; detecting the presence of the label in the sample, and identifying the labeled binding partner.

In some aspects, the binding partner is a target of interest and the disclosed methods can be used to identify a molecule of interest that binds to a specific target of interest. For example disclosed are methods of identifying a molecule of interest that binds to a binding partner comprising adding a labeling composition and a targeting composition to a sample, wherein the labeling composition comprises a ligandable tag, a linker, and a labeling enzyme, wherein the linker attaches the ligandable tag to the labeling enzyme, wherein the targeting composition comprises a ligand conjugated to a molecule of interest, wherein the ligand of the targeting composition is a ligand for the ligandable tag of the labeling composition; incubating the labeling composition and targeting composition with the sample under conditions that allow the molecule of interest to bind to a binding partner in the sample, wherein when the molecule of interest binds to a binding partner in the sample, wherein the labeling enzyme labels the binding partner; detecting the presence of the label in the sample, and thereby identifying the molecule of interest that binds to the binding partner.

In some aspects, the methods of identifying a binding partner can include all of the steps of the disclosed methods of detecting disclosed herein. In some aspects, the methods of identifying a binding partner can further comprise isolating the labeled binding partner from the molecule of interest prior to identifying a binding partner.

In some aspects, identifying the labeled binding partner can further comprise isolating any labeled molecules from the sample. In some aspects, the methods of identifying a binding partner allows for the removal of any non-specific artifacts present in a sample.

In some aspects, identifying the labeled binding partner comprises performing proteomics, mass spectrometry, immunoblotting, imaging, Homogeneous Time-Resolved Fluorescence (HTRF), mesoscale, Elisa, luminex, alphalisa, or nanopore based sequencing of peptides.

In some aspects, adding a labeling composition and a target composition to a sample can be performed simultaneously or consecutively.

In some aspects, incubating the labeling composition and target composition with the sample can occur for at least 0.1, 0.25. 0.5, 0.75, 1, 2, 3, 4, 5, 6, 7, 8, 9, 10, 12, 14, 16, 18, 20, 22, or 24 hours. In some aspects, the incubating can occur while the sample is rocking or mixing.

In some aspects, detecting the presence of the label in the sample can vary based on what labeling system is used. For example, if the label is biotin then the biotin labeled binding partner can be isolated with streptavidin and then detected using immunoassays and/or imaging.

In some aspects, the sample comprises the binding partner (i.e. target of interest). In some aspects, the sample is a cell culture, bodily fluid (e.g. blood, plasma, urine, saliva, etc.), tissue (e.g. brain, muscle, heart), or whole organism (e.g. zebrafish, fly, etc).

In some aspects, the labeling enzyme is any enzyme which can label a target of interest, such as a protein, for biochemical analysis. In some aspects, the labeling enzyme is a protein with ligase activity. In some aspects, the protein with ligase activity is a biotin ligase. Formlyglycine, sortase, transglutaminase, famesyltransferase, or lipoic acid ligase. In some aspects, a biotin ligase can be, but is not limited to, miniTurboID, TurboID, or BirA.

In some aspects, the ligandable tag in the labeling composition binds to its ligand in the target composition bringing together the labeling composition and the target composition. In some aspects, the ligandable tag is FKBP^{F36V}, Halo TAG, SNAp-tag, CLIp-TAG, LigandLink, or cutinase. Thus, in some aspects, the ligand (in the target composition) can be, Ortho-AP1867, Haloalkanes (e.g. JF549), 06-benzylguanine/06-benzylguanine derivatives/benzyl-2-chloro-6-aminopyrimidines, benzylcytosine derivatives, trimethoprim derivatives, or p-nitrophenyl phosphonate, respectively.

In some aspects, the targeting composition comprises a linker between the ligand and molecule of interest. In some aspects, the linker is a chemical linker.

In some aspects, the labeling composition comprises a linker between the ligandable tag and labeling enzyme. In some aspects, the linker is an amino acid based linker.

In some aspects of the disclosed method, the ligand of the targeting composition binds to the ligandable tag of the labeling composition. Meanwhile, the molecule of interest on the targeting composition binds to a binding partner (target of interest) in the sample. Due to the binding of the labeling composition and the target composition, the labeling enzyme on the labeling composition is in close enough proximity to the binding partner in the sample to result in the labeling enzyme attaching a label to the binding partner. In some aspects, any other molecules also bound to the binding partner can also be labeled by the labeling enzyme. In some aspects, this allows for detecting the binding partner and other molecules that interact with the binding partner. Once the presence of a binding partner is detected, the binding partner can be identified. In some aspects, the labeled binding partner can be isolated. For example, the label can be used to also isolate, such as using biotin label and isolating with streptavidin. In some aspects, once isolated, the binding partner can be analyzed using any known method for analyzing proteins or nucleic acids.

### 3. Methods of Screening

Disclosed are methods of screening a compound of interest comprising adding a labeling composition and a target composition to a sample, wherein the labeling composition comprises a ligandable tag conjugated to a labeling enzyme, wherein the target composition comprises a ligand and a molecule of interest, wherein the ligand of the target composition is a ligand for the ligandable tag of the labeling composition; incubating the labeling composition, target composition and a compound of interest with the sample under conditions that allow the molecule of interest to bind to a binding partner in the sample, wherein if the compound of interest does not interfere with the molecule of interest binding to a binding partner then the molecule of interest binds to a binding partner, and the labeling enzyme labels the binding partner, wherein if the compound of interest interferes with the molecule of interest binding to a binding partner then the molecule of interest does not bind to a binding partner and the labeling enzyme does not label the binding partner; detecting the presence or absence of the label in the sample. Thus, in some aspects, the presence of a label is indicative of a binding partner of a molecule of interest present in the sample, wherein the binding is not altered by the compound of interest. In some aspects, the absence of a label is indicative of a compound of interest interfering with the molecule of interest binding to a binding partner. Therefore, in some aspects, this screen can identify drugs that are involved in certain pathways.

In some aspects, if the compound of interest enhances binding of a molecule of interest to its binding partner, the disclosed method of screening would show enhanced binding (or higher levels of binding).

In some aspects, this method uses any of the labeling compositions and a target compositions disclosed herein.

In some aspects, incubating with the compound of interest can occur for at least 1, 2, 3, 4, 5, 6, 7, 8, 9, 10, 12, 14, 16, 18, 20, 22, or 24 hours.

In some aspects, the sample comprises the binding partner (i.e. target of interest). In some aspects, the sample is a cell culture, bodily fluid (e.g. blood, plasma, urine, saliva, etc.), tissue (e.g. brain, muscle, heart), or whole organism (e.g. zebrafish, fly, etc).

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed compositions.

### EXAMPLES

### A. Example 1: a Biotin Targeting Chimera (BioTAC) System to Map Small Molecule Interactomes In Situ

### 1. Introduction

Small molecule targeted therapies are a cornerstone of modern medicine, providing effective treatments for a wide range of diseases. However, the clinical effects of candidate therapies in patients can be highly variable, even when they bind the same therapeutic target with equal affinity. This divergent pharmacology can be due to uncharacterized off-target effects, combinatorial polypharmacology, or from small molecules inducing changes in the interactome of their protein targets through allosteric effects or ‘molecular glue’ effects. Despite the critical role of small molecules in drug discovery and development, there is a lack of comprehensive, network-scale profiling methods that inform on the cellular interactomes of small molecules.

Examples of small-molecule mediated interactome rewiring are well studied in cancer, where targeted therapies frequently induce functional changes in the complexation of their protein targets. For example, blockbuster cancer drugs, such as trametinib and lenalidomide, exert efficacy through the promotion of novel protein complexes, now known as ‘molecular glue’ pharmacology. Unanticipated protein complex rewiring is also a major cause of drug candidate failures, for example underpinning adverse effects of 1st generation RAF inhibitors in RAS driven tumors, and resistance to BET bromodomain inhibitors in triple-negative breast cancer and neuroblastoma. However, the discovery of interactome changes that mediate both drug efficacy and drug resistance has so far been serendipitous, as researchers investigate why certain drugs display unexpected pharmacology in the clinic. Despite their central importance, effects on target complexation remain uncharacterized for most protein ligands, representing a ‘blind spot’ in compound characterization workflows.

Current gold-standard technologies for unbiased target-ID are the chemical proteomic techniques photoaffinity labelling and microenvironment mapping (μMap) that use UV-light initiated diazirine photochemistry to label liganded proteins with affinity handles. Here, off-compete experiments with free drug, or comparison to a chemically matched negative control molecule facilitate data interpretation. However, in the intracellular context these techniques are directed towards detection of the primary target(s) only, due to the short half-life of the generated reactive carbene species which corresponds to a labelling radius of ˜6 nm. Here, the linker length between the diazirine or iridium photocatalyst and the drug dictates the labelling radius, and is therefore limited by cell permeability of the conjugate. As such, they have yet to be applied to map drug-bound complexes. Diazirine photochemistry approaches are also currently incompatible with in vivo applications.

To understand drug-induced interactome changes, affinity purification coupled to mass spectrometry (AP/MS) and proximity labeling coupled to mass spectrometry are commonly employed. Proximity labeling techniques such as BioID and TurboID are particularly advantageous in mapping interactomes. Proximity labeling methods have a labeling radius of up to 35 nm and can be used in live cells and animals. By fusing a target protein or localization tag with a proximity labeling enzyme proximity labeling can reliably detect transient, moderate affinity protein-complex interaction in situ due to the ability of biotin ligase to accumulate affinity tags on these protein partners over time. However, both techniques rely on a priori target knowledge, which is often incompletely understood for drug candidates, and the fusion of the target protein to either an affinity-tag or a proximity labeling enzyme, which can significantly impact interacting proteins. Therefore, they cannot be used for unbiased interactome-ID of small molecules. The outputs of these techniques are also typically large numbers of enriched proteins, due to their low stringency, making data interpretation and validation challenging.

To facilitate routine evaluation of ligand-target interactome changes induced by either inhibitors or molecular glues in a single experiment, a method was envisioned that combines the precision and unbiased nature of chemical proteomics with the sensitivity, whole-organism compatibility, and extended detection radius of proximity-labelling coupled to mass spectrometry. Here, the development of a ligand-guided miniTurboID method to accomplish these goals is reported, and it is benchmarked against gold-standard unbiased technologies for both target-ID and interactome-ID.

### 2. Results

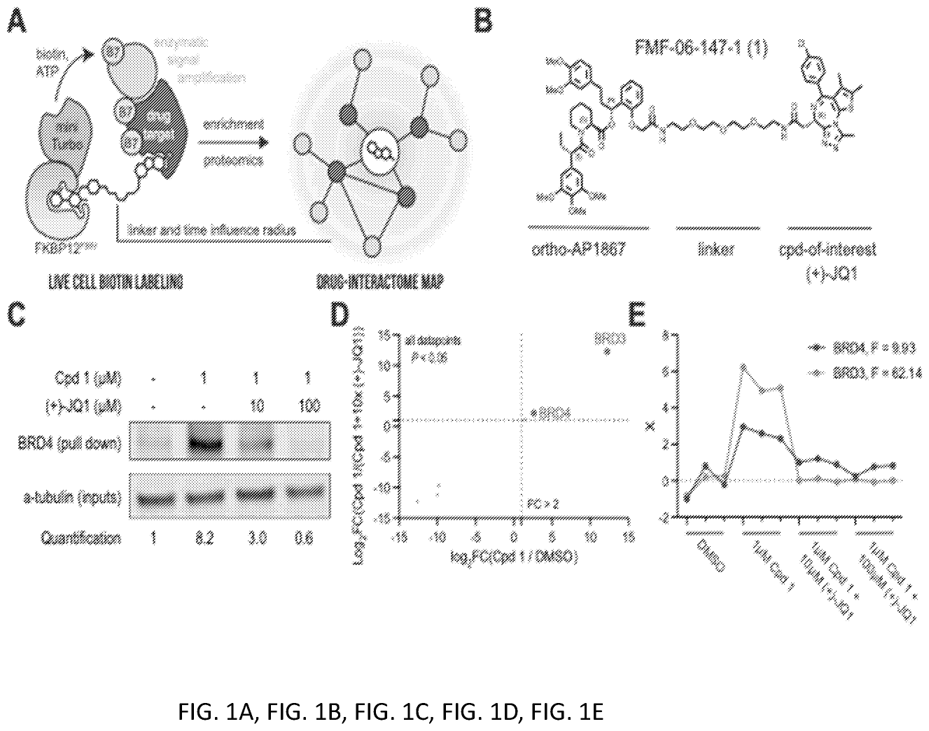

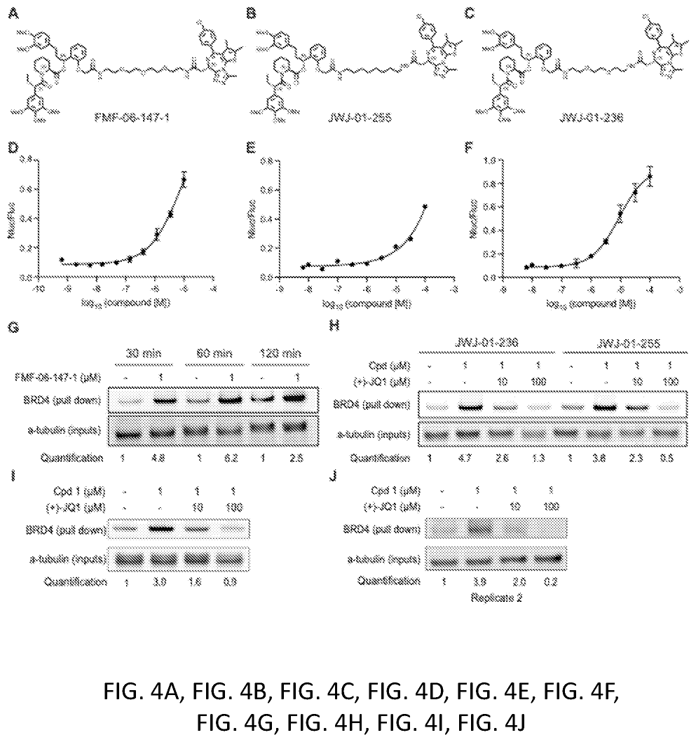

This method, named the biotin targeting chimera (BioTAC) system, uses bifunctional molecules composed of a compound-of-interest linked to selective FKBP12F36V recruiter orthoAP1867, to recruit a ligandable proximity labeling enzyme (mTurbo-FKBP12F36V) to compound-bound complexes enabling their biotinylation and subsequent affinity purification (FIG. TA-B). To benchmark the BioTAC system for accurate detection of the direct targets of ligands the well-characterized BET protein inhibitor (+)-JQ1, which potently binds BRD2, BRD3 and BRD4 (as well as the testes-specific BET protein BRDT), was selected as a test case. A series of bifunctional molecules consisting of (+)-JQ1 conjugated to orthoAP1687 were synthesized via a variable linker (FIG. 1B, FIG. 4A-C). (+)-JQ1-bifunctional molecule cell permeability was measured using an FKBP12F36V cellular target engagement assay adapted from Nabet et. al Nat Commun 11, 4687 (2020). Briefly, FKBP12F36V binding molecules compete with an FKBP12F36V targeting PROTAC (dTAG-13), for NLuc-FKBP12F36V occupancy, rescuing degradation and resulting in an increase in NLuc/FLuc (control) signal relative to DMSO-treated cells. All synthesized compounds were comparably cell permeable (FIG. 4D-E).

Next bifunctional molecule guided-proximity labeling experiments were performed using validated reagents. HEK293 cells transiently transfected with mTurbo-FKBP12F36V were treated with 100 μM biotin, 1 μM bifunctional (+)-JQ1 recruiters, and variable concentrations of free (+)-JQ1 to off-compete the bifunctional molecule and rescue biotinylation. Biotinylated proteins were isolated from cell lysates via streptavidin bead pulldown and analyzed by western blot for BRD4, a primary target of (+)-JQ1. Significant enrichment of BRD4 and a dose dependent decrease in BRD4 pulldown was observed in the presence of unconjugated (+)-JQ1 at all timepoints evaluated (FIG. 1C, FIG. 4G-J). Comparable activity was observed for all bifunctional analogues, indicating low sensitivity to linker length (FIG. 4G-J), FMF-01-147-1 (Cpd1) was selected for further characterization based on high BRD4-labeling and rescue at the 30 min time point (FIG. 1C).

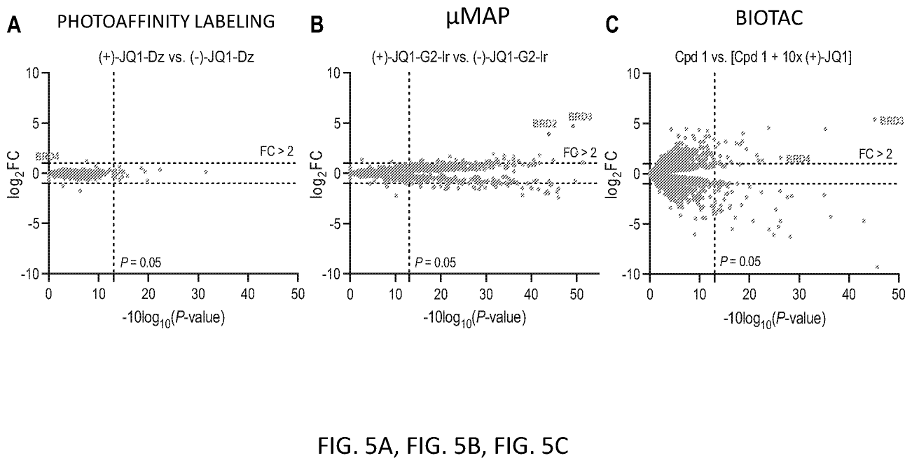

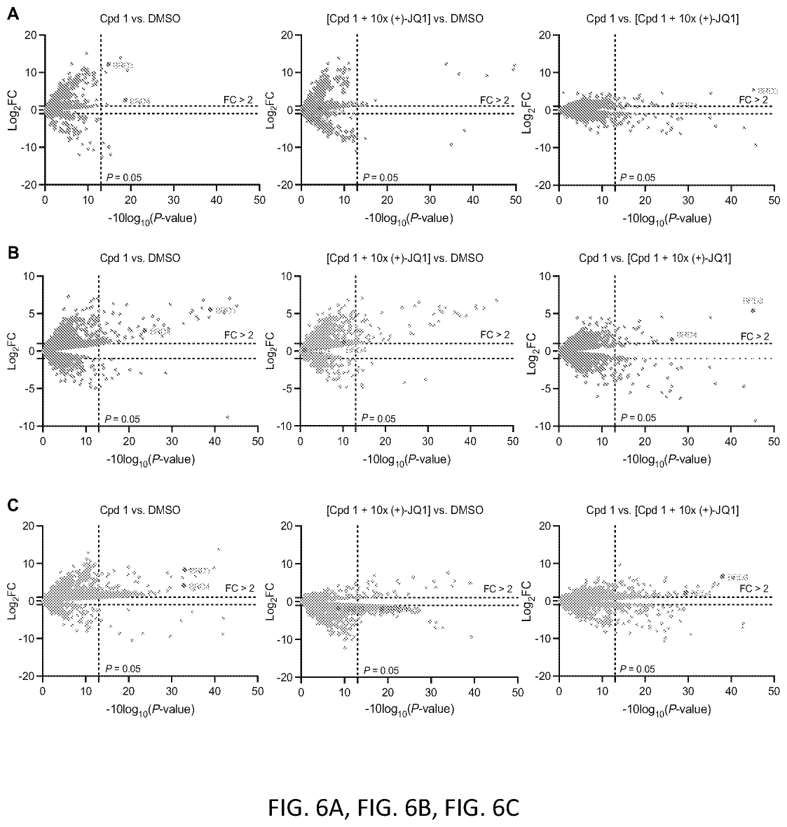

Having determined that the BioTAC system could identify BRD4 as a (+)-JQ1 target in focused screens, it was evaluated as an unbiased target-ID method. To identify direct binders of (+)-JQ1 proteome-wide, BioTAC proximity labelling experiments were performed as described above, followed by label-free mass spectrometry-based proteomic analysis of biotinylated proteins enriched following a 30 minute treatment with 100 μM biotin plus DMSO or 1 μM Cpd1, and then 1 μM Cpd1 off-competed by pre-treatment with 10 μM free (+)-JQ1. Highly selective enrichment of known (+)-JQ1 targets BRD3 and BRD4, comparable to published (+)-JQ1 μMapping and superior to published (+)-JQ1 photoaffinity labelling was observed (FIG. 1D-E, FIG. 5A-C, FIG. 6A-C).

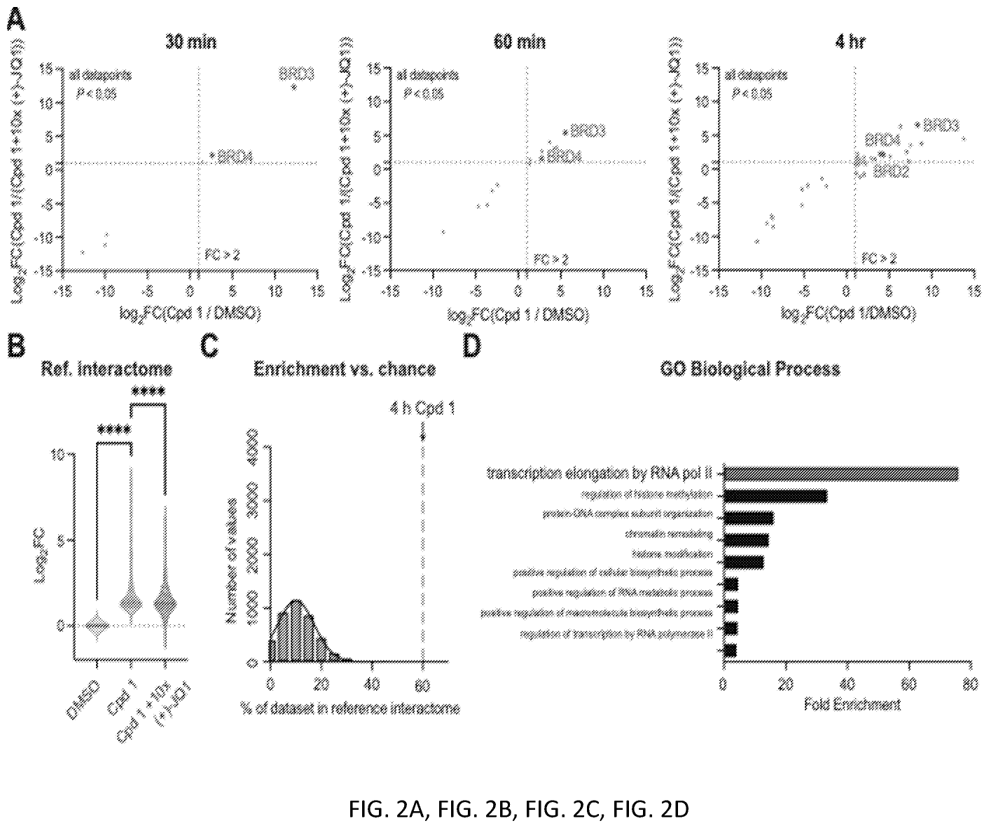

Having established conditions for determining the primary targets of small molecules using the BioTAC system, its utility in reading out the interactome sphere of (+)-JQ1 bound BET-proteins was next investigated. An advantage of the BioTAC system is the relatively long half-life of the activated biotin-AMP intermediate generated by TurboID, allowing the labelling radius to be increased up to 35 nm by extending the labelling time. To quantitively evaluate the ability of the BioTAC system to successfully enrich the known interactome of (+)-JQ1 bound BET proteins, time course BioTAC proximity labelling experiments were performed at the 1 h and 4 h time points, and evaluated streptavidin-enriched biotinylated proteins using mass spectrometry-based proteomic analysis (FIG. 2A-C, FIG. 6). Longer time points correlated with a greater number of proteins meeting the significance cut-offs (defined as FC>2, P<0.05) for both enrichment in Cpd1 vs. DMSO, and competition with free (+)-JQ1 in Cpd1 vs. Cpd1+10 μM (+)-JQ1, (FIG. 2A, FIG. 6). Known (+)-JQ1 direct targets BRD3 and BRD4 were significantly enriched and off-competed at all time points and BRD2 was also detected at the 4 hr timepoint. Additionally, identified hit proteins at 1-4 hrs also included many known BET interactors, such as proteins found in components of the Pol II productive elongation complex subunit P-TEFb, the TFILD complex, and the Nucleosome Remodeling Deacetylase (NuRD) complex, as well as known BRD4 direct-binder Histone H4. These observations indicated that direct interactors of (+)-JQ1 bound BRD2, 3, and 4 may be labeled. To test this hypothesis the hits were compared to an extensive BET protein interactome reference dataset identified using AP/MS and proximity labelling coupled to mass spectrometry with and without (+)-JQ1. The BioTAC system afforded significant enrichment and off-competition (P<0.0001, one-way ANOVA) of identified reference interactors at the 4 h time point (FIG. 2B). Next it was asked whether the inverse could be performed, enriching complex members identified by Lambert et al. Mol Cell 73, 621-638 e617 (2019) directly from the data without any a priori knowledge of known interactors. It was discovered that 60% of the statistically significant enriched and (+)-JQ1 rescued hits were also present in the Lambert dataset. This observation falls greater than 7 standard deviations above a random bootstrap analysis of the data without filtering for significant enrichment and (+)-JQ1 rescue (FIG. 2C). To account for the limitations of using one study, Gene Ontology (GO) Biological Process analysis of the hits was performed from the 4 hr timepoint, where transcriptional elongation by Pol II was significantly enriched (P<0.05), consistent with the known functions of BET protein containing complexes targeted by bromodomain inhibitors (FIG. 2D).

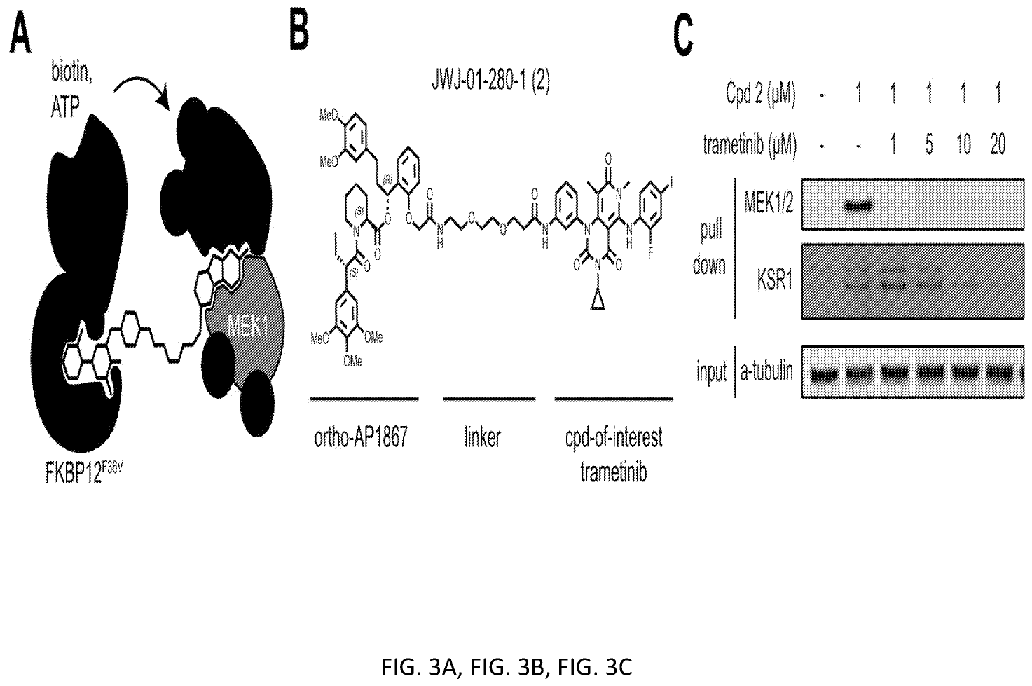

The paucity of practical methods for rapid, unbiased readout of small-molecule induced interactome changes has hindered the rational discovery and development of molecular glues. Molecular glues are small molecules which exert their function by binding to a primary target, and promoting or strengthening its interaction with a second protein through interactions at the protein-protein interface. Molecular glue discovery is an area of high biomedical interest due to the ability of molecular glues to target undruggable oncoproteins such as transcription factors, which are recalcitrant to traditional inhibitor discovery but can be neutralized by induced complexation and targeted degradation. However, as the binary affinity between a molecular glue and the second recruited target is low or non-existent in the absence of the primary target, molecular glue interactions are challenging to detect and screen for. Having rigorously benchmarked the performance of the BioTAC system using well-characterized (+)-JQ1, the BioTAC system was used to inform on complexes assembled by molecular glues (FIG. 3A). Trametinib was selected as a non-dergrader glue with which to benchmark the platform. Trametinib derives its clinical anti-cancer efficacy from promoting the interaction of its primary target MEKT/2 with KSR1, but has low affinity for KSR1 alone meaning this interaction was missed during clinical developments.

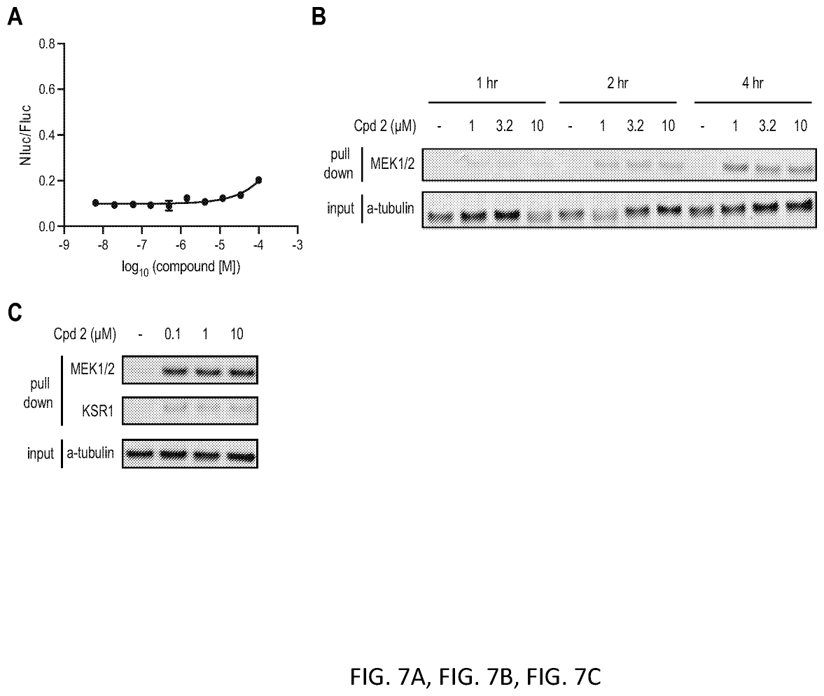

Bifunctional trametinib analogue JWJ-01-280/Cpd2 was synthesized, with a linker attachment informed by a reported trametinib-derived BRET probe named Tram-bo (FIG. 3B). The cell permeability of Cpd2 was evaluated as described above, which was less cell permeable than the (+)-JQ1 derivatives (FIG. 7A). Nevertheless, Cpd2 supported efficient MEK1 labelling, and following dose and time point optimization (FIG. 7B-C). The ability of the BioTAC system to detect both known interactors of trametinib was evaluated by Western blot, as described above. The MEK1:trametinib:KSR1 complex was detected using the BioTAC system following a 4 hr treatment with 1 μM Cpd2, and dose-dependent competition in the presence of trametinib of both KSR1 and MEK1 by immunoblot (FIG. 3B). Together, these data demonstrate the ability of the BioTAC system to identify the binding partners of non-degrader molecular glues.

### 3. Discussion

Methods for the routine measurement of drug-target interactomes are lacking, hiding the mechanism of action of numerous small molecules from view. Even though interactome remodelling in response to small molecule drugs is a common phenomenon that mediates drug efficacy and resistance, little progress has been made in identifying and characterizing such events. Here the BioTAC system is reported, which can identify the direct target of a small molecule, as well as its complexed proteins with high confidence. Enrichment of the reported interactomes of the epigenetic inhibitor (+)-JQ1 and the molecular glue trametinib was reported, but this approach is theoretically applicable to any small molecule of interest that can be functionalized with a linker.

Next, the BioTAC system is shown that it can identify molecular glue pairs that previously evaded detection, in a single experiment using trametinib, MEK1/2, KSR1, as a model system. The discovery and detection of molecular glue interactions is notoriously challenging to evaluate. Recently, elegant workflows consisting of mechanism-based screening in wild-type and hypo-NEDDylated cells, followed by multi-omic target deconvolution have been described for the discovery of cullin-ring ligase (CRL)-recruiting molecular glue degraders. To identify non-degrader molecular glues, size exclusion chromatography coupled to mass spectrometry has been used in combination with activity-based protein profiling to screen electrophilic compound libraries, yielding covalent stabilizers and disruptors of protein-protein interactions. However, these approaches require resource intensive multi-omic workflows, and are limited to covalent glues, or CRL-mediated degradation mechanisms. The BioTAC system can be used in a screening mode, for unbiased profiling of putative glue libraries.

The BioTAC system uses a universal recruitable biotin ligase chimera, enabling an unbiased approach and facilitating rapid application. Bifunctional molecules for investigating any drug-of-interest are synthesised in one step from a common ortho-AP1867 precursor using robust coupling chemistries. Finally, the enrichment, mass spectrometry and data analysis methods are adapted from standard protocols in proximity labelling already performed by most proteomics core facilities. These features make BioTAC system readily accessible for broad application. During the preparation of this manuscript, a preprint describing a related approach for identifying targets of small molecules via SNAP- and Halo-tagging of TurboID was also disclosed. Whilst these systems differ from the BioTAC system in their reported specificity, their successful implementation across a range of ligands highlights the robustness of using proximity labelling to interrogate small molecule targets.

In the long term, building community-wide knowledge around how small molecule drugs alter their target proteins complexation will lay the foundation for the rational design of drug target interactome profiles, to combat drug resistance, and enable wider targeting of the undruggable proteome.

### 4. Methods

i. Cell Culture

The following cell lines were employed in this study: HEK293 (source: ATCC #CRL-1573, media: DMEM with 10% FBS (vendor) and 1% Penicillin-Streptomycin (vendor)). All cell lines were maintained in 37° C. and 5% CO2 incubators and routinely tested negative for mycoplasma contamination using the MycoAlert Kit (Lonza).

ii. BioTAC Plasmids

pCDNA3.1(+) miniTurboID-6xGGSGlinker-FKBPF36V-2xHA.

DNA encoding the BioTAC construct (below) was synthesized at GenScript and cloned into the MCS of pCDNA3.1(+) by NEBuilder HiFi DNA Assembly (NEB #E2621).

(SEQ ID NO: 4)
ATGGCTCGGGATCCACCGGTCGCCACCATGATCCCGCTGCTGAACGCTAA
ACAGATTCTGGGACAGCTGGACGGCGGGAGCGTGGCAGTCCTGCCTGTGG
TCGACTCCACCAATCAGTACCTGCTGGATCGAATCGGCGAGCTGAAGAGT
GGGGATGCTTGCATTGCAGAATATCAGCAGGCAGGGAGAGGAAGCAGAGG
GAGGAAATGGTTCTCTCCTTTTGGAGCTAACCTGTACCTGAGTATGTTTT
GGCGCCTGAAGCGGGGACCAGCAGCAATCGGCCTGGGCCCGGTCATCGGA
ATTGTCATGGCAGAAGCGCTGCGAAAGCTGGGAGCAGACAAGGTGCGAGT
CAAATGGCCCAATGACCTGTATCTGCAGGATAGAAAGCTGGCAGGCATCC
TGGTGGAGCTGGCCGGAATAACAGGCGATGCTGCACAGATCGTCATTGGC
GCCGGGATTAACGTGGCTATGAGGCGCGTGGAGGAAAGCGTGGTCAATCA
GGGCTGGATCACACTGCAGGAAGCAGGGATTAACCTGGACAGGAATACTC
TGGCCGCTATGCTGATCCGAGAGCTGCGGGCAGCCCTGGAACTGTTCGAG
CAGGAAGGCCTGGCTCCATATCTGTCACGGTGGGAGAAGCTGGATAACTT
CATCAATAGACCCGTGAAGCTGATCATTGGGGACAAAGAGATTTTCGGGA
TTAGCCGGGGGATTGATAAACAGGGAGCCCTGCTGCTGGAACAGGACGGA
GTTATCAAACCCTGGATGGGCGGAGAAATCAGTCTGCGGTCTGCCGAAAA
GGCTAGCGGTGGTTCTGGTGGCGGCAGTGGGGGGGGGTCCGGTGGCGGAT
CAGGTGGTGGAAGTGGCGGTGGCTCAGGTGGAGTGCAGGTGGAAACCATC
TCCCCAGGAGACGGGCGCACCTTCCCCAAGCGCGGCCAGACCTGCGTGGT
GCACTACACCGGGATGCTTGAAGATGGAAAGAAAGTTGATTCCTCCCGGG
ACAGAAACAAGCCCTTTAAGTTTATGCTAGGCAAGCAGGAGGTGATCCGA
GGCTGGGAAGAAGGGGTTGCCCAGATGAGTGTGGGTCAGAGAGCCAAACT
GACTATATCTCCAGATTATGCCTATGGTGCCACTGGGCACCCAGGCATCA
TCCCACCACATGCCACTCTCGTCTTCGATGTGGAGCTTCTAAAACTGGAA
GGCGGCTACCCCTACGACGTGCCCGACTACGCCGGCTATCCGTATGATGT
CCCGGACTATGCAGGCTAA

iii. Immunoblotting

0.6 million HEK293 cells were plated into wells containing 1 mL of complete media (DMEM, 10% FBS, 1% pen/strep) and incubated overnight. Cells were transiently transfected with 100 μL of OptiMEM media containing 1 μg of BioTAC plasmid and 3 μL of TransIT-2020 transfection reagent (Mirus, MIR5400) for 24 hr. Following a media change, cells were pre-treated with parental molecules or DMSO for 10 min, then treated with bifunctional compound for 10 min, and finally labeled in 100 μM biotin for the indicated time. Cells were washed twice with DPBS then harvested into 1.5 mL tubes. Cells were lysed on ice for 15 min in 100 uL RIPA lysis buffer (50 mM Tris pH 8, 150 mM NaCl, 0.1% SDS, 0.5% sodium deoxycholate, 1% Triton X-100) containing fresh HALT inhibitor cocktail (Thermo, 78442), 100 mM PMSF, and 125 U/mL benzonase (MilliporeSigma, 70746-4), then clarified by centrifuging at 16,000×g for 10 min at 4° C. Protein concentration was determined by BCA assay (Thermo, 23225), then 100 ug protein was rotated overnight at 4° C. with 8 μL of streptavidin magnetic beads (Thermo, 88817) that had been prewashed twice in RIPA lysis buffer. Flowthrough was collected, and beads were washed twice in RIPA lysis buffer, once in 1 μM KCl, once quickly in 0.1 μM sodium carbonate, once quickly in 2 μM urea 50 mM Tris pH 8, and finally twice in RIPA lysis buffer. Following the final wash, biotinylated proteins were eluted by boiling the beads in 1× NuPage LDS sample buffer (Invitrogen, NP0007) containing 2 mM biotin and 20 mM DTT at 95° C. for 10 min. Input and flowthrough samples were prepared in 1× NuPage LDS sample buffer. SDS-PAGE samples were run on a 4-12% bis-tris precast gel (Invitrogen, NW04125BOX) for 45 min at 180V, then transferred onto a nitrocellulose membrane (Bio-Rad, 1620112) for 90 min at 45V. The membrane was blocked in 5% non-fat dry milk in TBST for 1 hr then incubated in 1:1000 BRD4 anti-rabbit (Bethyl Laboratories, A301-985A-M) and 1:1000 alpha-tubulin anti-mouse (Cell Signaling Technology, 3873S)) overnight at 4 C. Membrane was washed three times in TBST then incubated in 1:10000 DyLight 680 anti-mouse IgG (Cell Signaling Technology, 5470S) and 1:10000 DyLight 800 anti-rabbit IgG (Cell Signaling Technology, 5151S) for 1 hr at RT. Membrane was washed three times in TBST then imaged with a ChemiDoc (Bio-Rad).

iv. Quantitative Proteomics

9 million HEK293 cells were plated into 15 cm dishes containing 12 mL of complete media (DMEM, 10% FBS, 1% pen/strep) and incubated overnight. Cells were transiently transfected with 1 mL of OptiMEM media containing 10 μg of BioTAC plasmid and 30 μL of TransIT-2020 transfection reagent for 24 hr. Following a media change, cells were optionally pre-treated with parental molecules for 10 min, then treated with bifunctional compound for 10 min, and finally labeled in 100 μM biotin for 30 min, 1 hr, or 4 hrs. Cells were washed 5 times with DPBS then harvested by scraping on ice into 1.5 mL tubes. Cells were lysed in 1.2 mL RIPA lysis buffer (50 mM Tris pH 8, 150 mM NaCl, 0.1% SDS, 0.5% sodium deoxycholate, 1% Triton X-100) containing fresh HALT inhibitor cocktail, 100 mM PMSF, and 125 U/mL benzonase. Cells were further lysed by sonicating with 15×1 second pulses at 40% power then chilled on ice for 15 min. Lysates were clarified by centrifuging at 16,000×g for 10 min and protein concentration determined by BCA assay. 3 mg protein was rotated ovemight at 4° C. with 250 μL of streptavidin magnetic beads that had been prewashed twice in RIPA lysis buffer. Beads were washed twice in RIPA lysis buffer, once in 1 μM KCl, once quickly in 0.1 μM sodium carbonate, once quickly in 2 μM urea 50 mM Tris pH 8, twice in RIPA lysis buffer, and finally 4 times in 50 mM Tris pH 8. Following the final wash, beads were transferred to clean tubes and frozen at −80° C.

Beads were resuspended in TNE buffer (50 mM Tris pH 8.0, 100 mM NaCl, 1 mM EDTA), then RapiGest SF reagent (Waters Corp.) was added to a final concentration of 0.1% and samples were boiled for 5 min. Samples were reduced with 1 mM TCEP (Tris (2-carboxyethyl) phosphine) at 37° C. for 30 min, then samples were carboxymethylated with 0.5 mg/ml of iodoacetamide for 30 min at 37° C. followed by neutralization with 2 mM TCEP. Samples were digested with trypsin (1:50 trypsin:protein ratio) overnight at 37° C. RapiGest SF was degraded and removed by treating the samples with 250 mM HCl at 37° C. for 1 h followed by centrifugation at 16,000×g for 30 min at 4° C. The soluble fraction was then added to a new tube and the peptides were extracted and desalted using C18 desalting columns (Thermo Scientific, PI-87782). Peptides were quantified using BCA assay and a total of 1 μg of peptides were injected for LC-MS analysis.

Trypsin-digested peptides were analyzed by ultra high pressure liquid chromatography (UPLC) coupled with tandem mass spectroscopy (LC-MS/MS) using nano-spray ionization. The nanospray ionization experiments were performed using a Orbitrap fusion Lumos hybrid mass spectrometer (Thermo) interfaced with nano-scale reversed-phase UPLC (Thermo Dionex UltiMate™ 3000 RSLC nano System) using a 25 cm, 75-micron ID glass capillary packed with 1.7-μm C18 (130) BEH™ beads (Waters corporation). Peptides were eluted from the C18 column into the mass spectrometer using a linear gradient (5-80%) of ACN (Acetonitrile) at a flow rate of 375 μl/min for 1.5 h. The buffers used to create the ACN gradient were: Buffer A (98% H2O, 2% ACN, 0.1% formic acid) and Buffer B (100% ACN, 0.1% formic acid). Mass spectrometer parameters are as follows an MS1 survey scan using the orbitrap detector, mass range (m/z): 400-1500 (using quadrupole isolation), 120000 resolution setting, spray voltage of 2200 V. Ion transfer tube temperature of 275° C., AGC target of 400000, and maximum injection time of 50 ms. MS1 survey scan was followed by data dependent scans using top speed for most intense ions, with charge state set to only include+2-5 ions, and 5 second exclusion time, while selecting ions with minimal intensities of 50000, in which the collision event was carried out in the high energy collision cell (HCD Collision Energy of 30%), and the fragment masses where analyzed in the ion trap mass analyzer (With ion trap scan rate of turbo, first mass m/z was 100, AGC Target 5000 and maximum injection time of 35 ms). Protein identification was carried out using Peaks Studio 8.5 (Bioinformatics solutions Inc.) with a 1% FDR cutoff. Data was imputed to remove missing values and DMSO normalized. Data was log 2 transformed and median-MAD scaled and a two-sample moderated T test with nominal P value=0.05 was used to filter for significant proteins.

General Methods for making bifunctional molecules to be used in the disclosed systemsUnless otherwise noted, reagents and solvents were obtained from commercial suppliers and were used without further purification. ¹H NMR spectra were recorded on 500 MHz Bruker Avance III spectrometer or 500 MHz JEOL ECA 500 spectrometer and chemical shifts are reported in parts per million (ppm, δ) downfield from tetramethylsilane (TMS). Coupling constants (J) are reported in Hz. Spin multiplicities are described as s (singlet), br (broad singlet), d (doublet), t (triplet), q (quartet), and m (multiplet). Mass spectra were obtained on a Waters Acquity UPLC/MS. Preparative HPLC was performed on a Waters Sunfire C18 column (19 mm×50 mm, 5 μM) using a gradient of 15-95% methanol in water containing 0.05% trifluoroacetic acid (TFA) over 22 min (28 min run time) at a flow rate of 20 mL/min. Assayed compounds were isolated and tested as TFA salts. Purities of assayed compounds were in all cases greater than 95%, as determined by reverse-phase HPLC analysis.

### a. tert-Butyl (S)-(1-(4-(4-chlorophenyl)-2,3,9-trimethyl-6H-thieno[3,2-f][1,2,4]triazolo[4,3-a][1,4]diazepin-6-yl)-2-oxo-6,9,12-trioxa-3-azatetradecan-14-yl)carbamate (2)

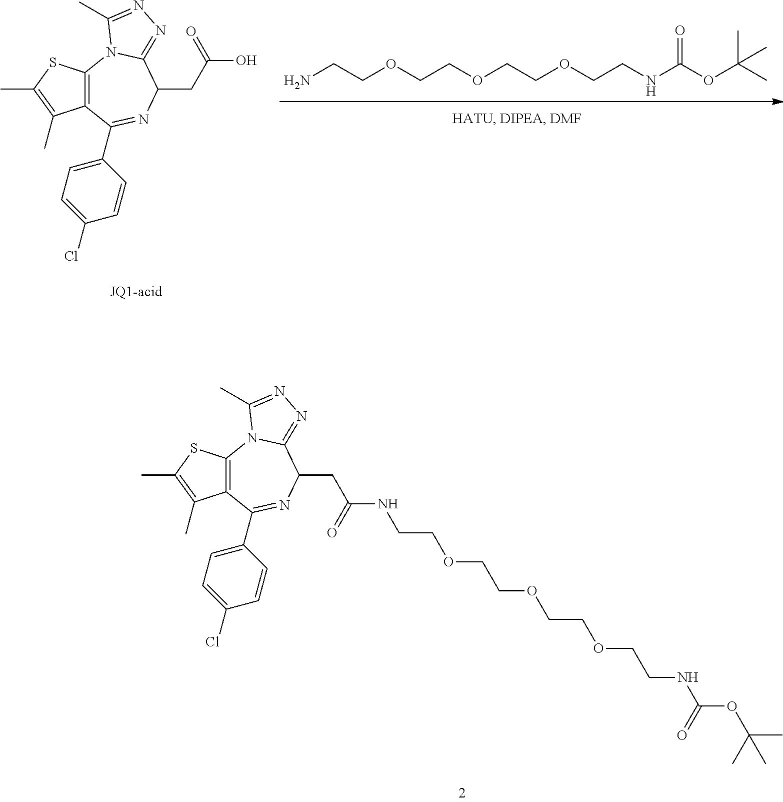 [SMILES: Cc1sc2c(c1C)C(c1ccc(Cl)cc1)=NC(CC(=O)O)c1nnc(C)n1-2 ; CC(C)(C)OC(=O)NCCOCCOCCOCCN ; Cc1sc2c(c1C)C(c1ccc(Cl)cc1)=NC(CC(=O)NCCOCCOCCOCCNC(=O)OC(C)(C)C)c1nnc(C)n1-2]

JQ1-acid (25 mg, 0.06 mmol), tert-butyl (2-(2-(2-(2-aminoethoxy)ethoxy)ethoxy)ethyl)carbamate (20 mg, 0.066 mmol), HATU (28 mg, 0.07 mmol), DIPEA (35 μL, 0.18 mmol) were dissolved in DMF (2 mL) and stirred at rt for 12 h. The reaction mixture was diluted in conc. aq. sodium bicarbonate (10 mL) and extracted with DCM (3×10 mL). The organics were combined, dried over MgSO₄, filtered and concentrated in vacuo. The crude product was purified via flash column chromatography (DCM:MeOH) to yield the title compound (27 mg, 0.04 mmol, 63%) as a colorless oil. MS (ESI) m/z 676 (M+H)⁺.

### b. (S)-N-(2-(2-(2-(2-aminoethoxy)ethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide (3)

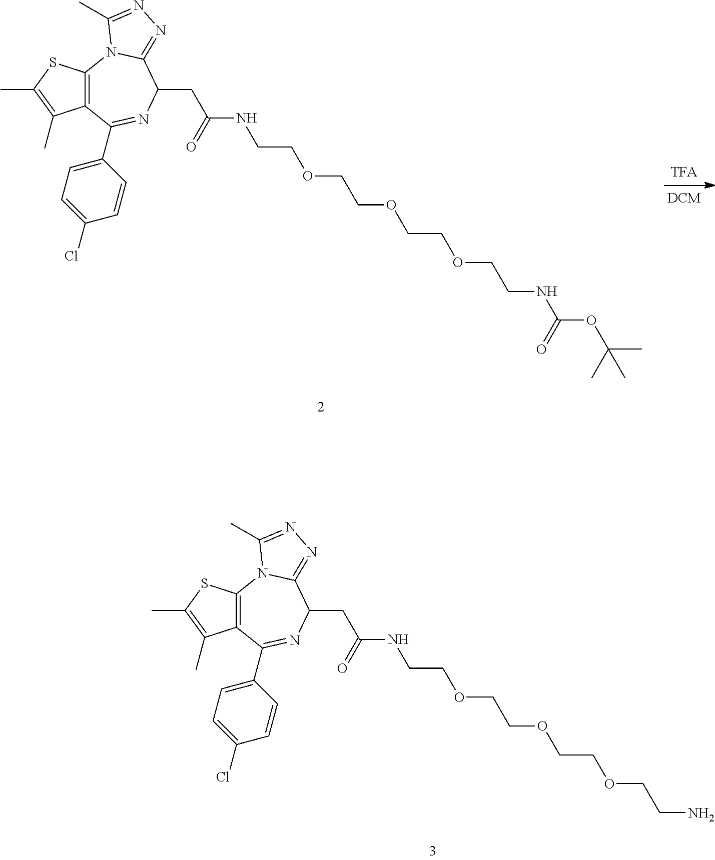

Compound 2 (27 mg, 0.04 mmol) was dissolved in 2 mL DCM and 0.5 mL TFA, and stirred at rt for 2 h. The reaction mixture was concentrated in vacuo and used without further purification (quant.). MS (ESI) m/z 576 (M+H)⁺.

### c. (R)-1-(2-((17-((S)-4-(4-Chlorophenyl)-2,3,9-trimethyl-6H-thieno[3,2-f][1,2,4]triazolo[4,3-a][1,4]diazepin-6-yl)-2,16-dioxo-6,9,12-trioxa-3,15-diazaheptadecyl)oxy)phenyl)-3-(3,4-dimethoxyphenyl)propyl (S)-1-((S)-2-(3,4,5-trimethoxyphenyl)butanoyl)piperidine-2-carboxylate (FM-06-147-1)

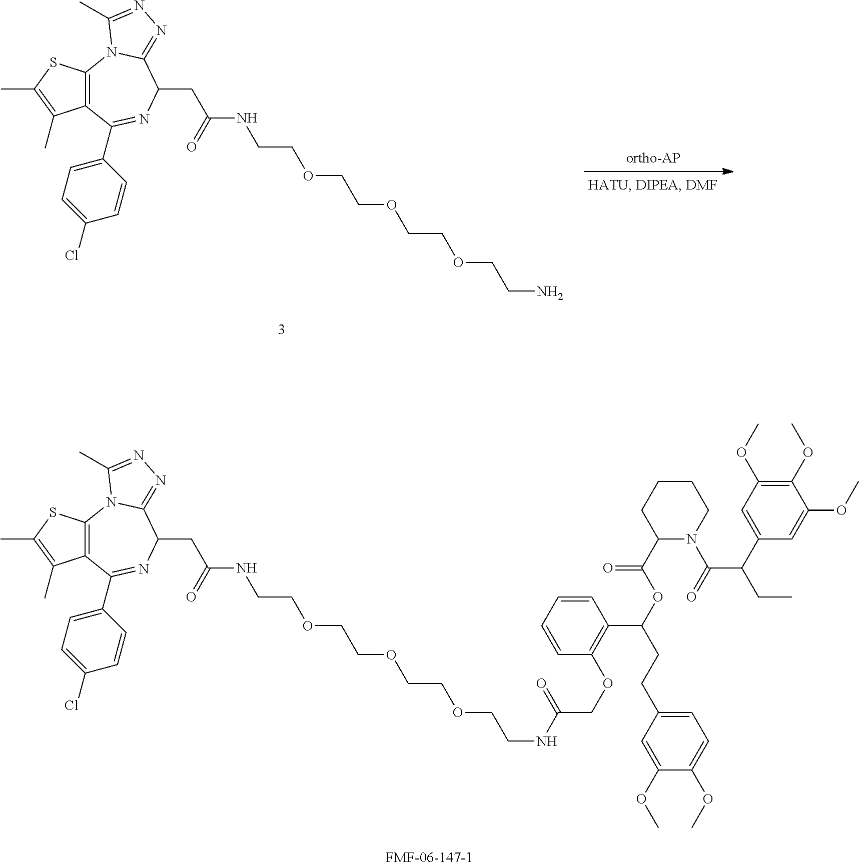

Compound 3 (25 mg, 0.04 mmol), ortho-AP (28 mg, 0.04 mmol), HATU (19 mg, 0.05 mmol), DIPEA (22 μL, 0.12 mmol) were dissolved in DMF (2 mL) and stirred at rt for 16 h. The reaction mixture was filtered and purified by HPLC to afford the title compound as a TFA salt (24 mg, 0.017 mmol), as a white solid. MS (ESI) m/z 626 (M+2H)²⁺/2. ¹H NMR (500 MHz, DMSO-d₆) δ 8.27 (t, J=5.7 Hz, 1H), 7.84 (t, J=5.7 Hz, 1H), 7.51-7.24 (m, 5H), 7.20 (ddd, J=8.6, 6.1, 3.0 Hz, 1H), 7.06-6.72 (m, 5H), 6.72-6.61 (m, 2H), 6.56 (s, 2H), 6.04 (dd, J=8.2, 4.7 Hz, 1H), 5.36-5.31 (m, 1H), 4.60-4.40 (m, 3H), 4.05 (d, J=13.4 Hz, 1H), 3.87 (dd, J=8.1, 6.5 Hz, 1H), 3.75 (s, 1H), 3.71 (d, J=9.1 Hz, 6H), 3.64 (s, 1H), 3.56 (d, J=8.0 Hz, 7H), 3.52-3.49 (m, 4H), 3.47 (q, J=2.2 Hz, 2H), 3.46-3.39 (m, 3H), 3.34-3.17 (m, 5H), 2.60 (d, J=1.3 Hz, 3H), 2.41 (s, 4H), 2.19-2.13 (m, 2H), 2.10-1.84 (m, 2H), 1.65-1.54 (m, 12H), 1.41-1.15 (m, 2H), 0.81 (t, J=7.3 Hz, 2H), 0.74 (t, J=7.3 Hz, 1H).

### d. tert-Butyl (S)-(8-(2-(4-(4-chlorophenyl)-2,3,9-trimethyl-6H-thieno[3,2-f][1,2,4]triazolo[4,3-a][1,4]diazepin-6-yl)acetamido)octyl)carbamate (4)

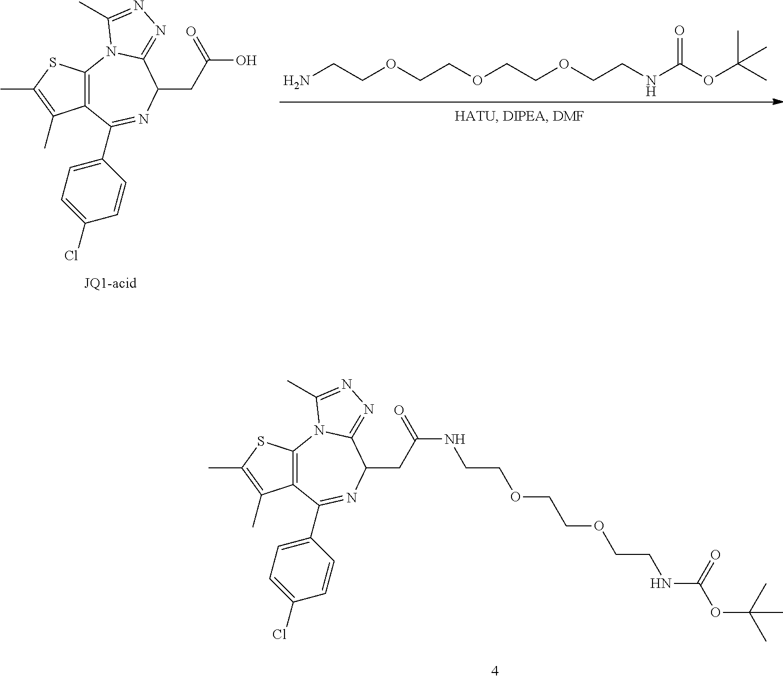 [SMILES: Cc1sc2c(c1C)C(c1ccc(Cl)cc1)=NC(CC(=O)O)c1nnc(C)n1-2 ; CC(C)(C)OC(=O)NCCOCCOCCOCCN ; Cc1sc2c(c1C)C(c1ccc(Cl)cc1)=NC(CC(=O)NCCOCCOCCNC(=O)OC(C)(C)C)c1nnc(C)n1-2]

JQ1-acid (25 mg, 0.062 mmol), HATU (28 mg, 0.075 mmol), DIPEA (33 uL, 0.19 mmol), and tert-butyl (2-(2-(2-aminoethoxy)ethoxy)ethyl)carbamate (17 mg, 0.069 mmol) were charged into a 4 mL vial. After adding 1 mL DMF, the solution was stirred for 2 hours. Upon the completion of the coupling reaction indicated by LCMS, the material was partitioned between DCM (15 mL) and brine (15 mL), and transferred to a separatory funnel. The layers were separated, and the aqueous phase was further extracted with DCM (15 mL). The organic extracts were dried over magnesium sulfate, filtered, and concentrated to dryness. The residue was purified by silica gel chromatography to yield 37 mg (95%) of the title compound. ¹H NMR (500 MHz, Methanol-d₄) δ 7.44 (d, J=8.3 Hz, 2H), 7.39 (d, J=8.7 Hz, 2H), 4.61 (dd, J=8.9, 5.3 Hz, 1H), 3.61 (ddd, J=11.9, 6.9, 3.5 Hz, 6H), 3.50 (t, J=5.7 Hz, 2H), 3.46-3.42 (m, 2H), 3.31 (d, J=5.2 Hz, 1H), 3.27 (s, 2H), 3.20 (t, J=5.6 Hz, 2H), 2.68 (s, 3H), 2.43 (s, 3H), 1.68 (s, 3H), 1.40 (s, 9H), 1.29 (dd, J=12.4, 6.3 Hz, 1H). MS (ESI) m/z 631.4 (M+H)⁺.

### e. (S)-N-(2-(2-(2-Aminoethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide (5)

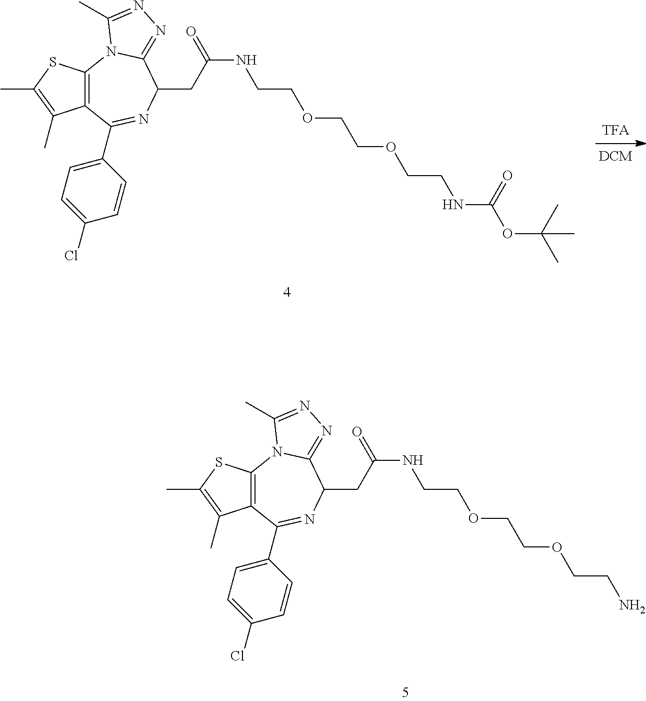

Compound 4 (37 mg, 0.06 mmol) was dissolved in 2 mL DCM and 0.5 mL TFA, and stirred at rt for 2 h. The reaction mixture was concentrated in vacuo and used without further purification (quant.). MS (ESI) m/z 531.4 (M+H)⁺.

### f. (R)-1-(2-((14-((S)-4-(4-Chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-3-(3,4-dimethoxyphenyl)propyl (S)-1-((S)-2-(3,4,5-trimethoxyphenyl)butanoyl)piperidine-2-carboxylate (JWJ-01-236)

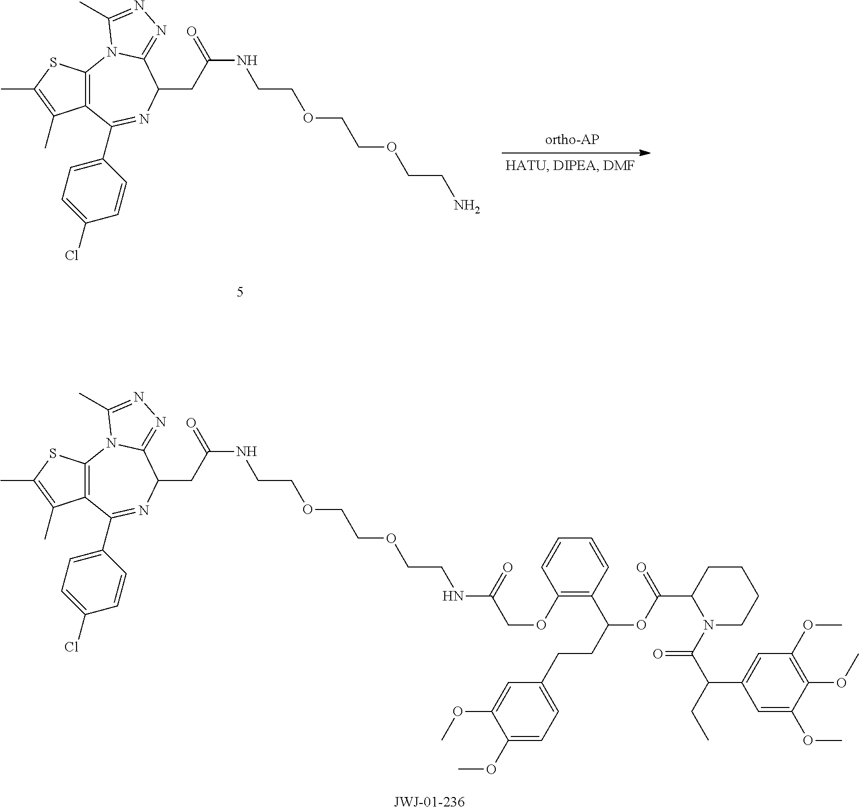

Compound 5 (37 mg, 0.05 mmol), ortho-AP (36 mg, 0.05 mmol), HATU (22 mg, 0.06 mmol), DIPEA (25 μL, 0.14 mmol) were dissolved in DMF (2 mL) and stirred at rt for 16 h. The reaction mixture was filtered and purified by HPLC to afford the title compound as a while solid (1.6 mg, 2.6%) with 99% purity. ¹H NMR (500 MHz, DMSO-d₆) δ 8.27 (s, 1H), 7.85 (t, J=5.7 Hz, 1H), 7.47 (d, J=8.7 Hz, 2H), 7.41 (d, J=8.2 Hz, 2H), 7.19 (ddt, J=8.7, 6.5, 3.3 Hz, 1H), 6.88-6.76 (m, 4H), 6.74 (d, J=2.0 Hz, 1H), 6.66-6.60 (m, 2H), 6.55 (s, 2H), 6.02 (dd, J=8.3, 4.7 Hz, 1H), 5.32 (t, J=4.2 Hz, 1H), 4.50 (q, J=5.0 Hz, 3H), 4.06-4.03 (m 2H), 3.87 (s, 2H), 3.71 (s, 3H), 3.52-3.47 (m, 6H), 3.42 (q, J=6.1 Hz, 5H), 3.26 (tdd, J=16.4, 12.6, 8.8 Hz, 8H), 2.58 (s, 6H), 2.40 (s, 4H), 2.15 (d, J=14.0 Hz, 1H), 2.02-1.83 (m, 3H), 1.66-1.50 (m, 8H), 1.31-1.20 (m, 4H), 0.76 (dt, J=45.9, 7.3 Hz, 3H). MS (ESI) m/z 1206.6 (M+H)⁺.

### g. tert-Butyl (S)-(8-(2-(4-(4-chlorophenyl)-2,3,9-trimethyl-6H-thieno[3,2-f][1,2,4]triazolo[4,3-a][1,4]diazepin-6-yl)acetamido)octyl)carbamate (6)

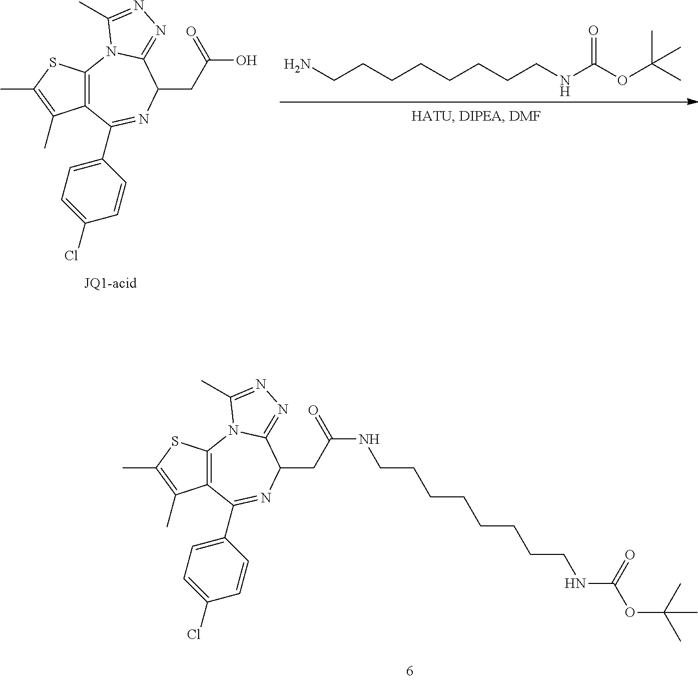 [SMILES: Cc1sc2c(c1C)C(c1ccc(Cl)cc1)=NC(CC(=O)O)c1nnc(C)n1-2 ; CC(C)(C)OC(=O)NCCCCCCCCN ; Cc1sc2c(c1C)C(c1ccc(Cl)cc1)=NC(CC(=O)NCCCCCCCCNC(=O)OC(C)(C)C)c1nnc(C)n1-2]

JQ1-acid (15 mg, 0.037 mmol), HATU (14 mg, 0.056 mmol), DIPEA (20 uL, 0.11 mmol), and tert-butyl (8-aminooctyl)carbamate (11 mg, 0.045 mmol) were charged into a 4 mL vial. After adding 1 mL DMF, the solution was stirred for 2 hours. Upon the completion of the coupling reaction indicated by LCMS, the material was partitioned between DCM (15 mL) and brine (15 mL), and transferred to a separatory funnel. The layers were separated, and the aqueous phase was further extracted with DCM (15 mL). The organic extracts were dried over magnesium sulfate, filtered, and concentrated to dryness. The residue was purified by silica gel chromatography to yield 23 mg (99%) of the title compound, MS (ESI) m/z 627.4 (M+H)⁺.

### h. (S)-N-(8-Aminooctyl)-2-(4-(4-chlorophenyl)-2,3,9-trimethyl-6H-thieno[3,2-f][1,2,4]triazolo[4,3-a][1,4]diazepin-6-yl)acetamide (7)

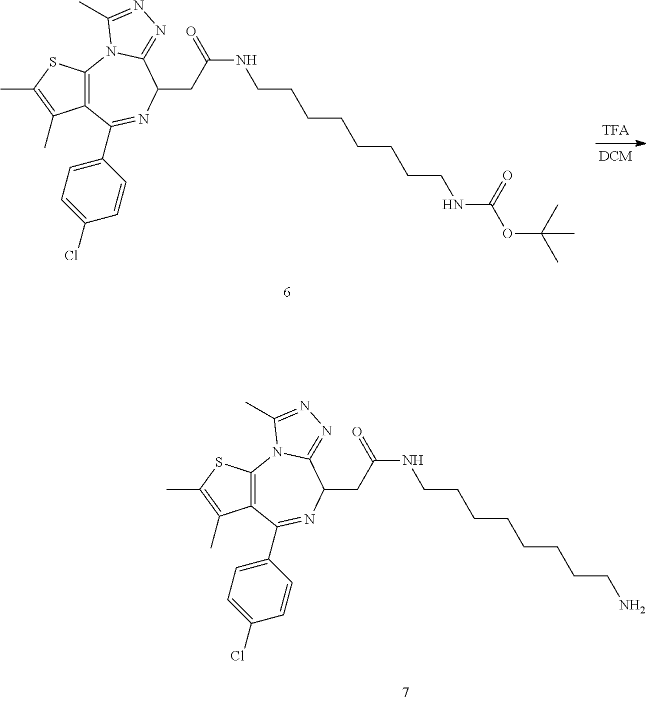

Compound 6 (23 mg, 0.04 mmol) was dissolved in 2 mL DCM and 0.5 mL TFA, and stirred at rt for 2 h. The reaction mixture was concentrated in vacuo and used without further purification (quant.). MS (ESI) m/z 527.3 (M+H)⁺.

### i. (R)-1-(2-(2-((8-(2-((S)-4-(4-Chlorophenyl)-2,3,9-trimethyl-6H-thieno[3,2-f][1,2,4]triazolo[4,3-a][1,4]diazepin-6-yl)acetamido)octyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (JWJ-01-255)

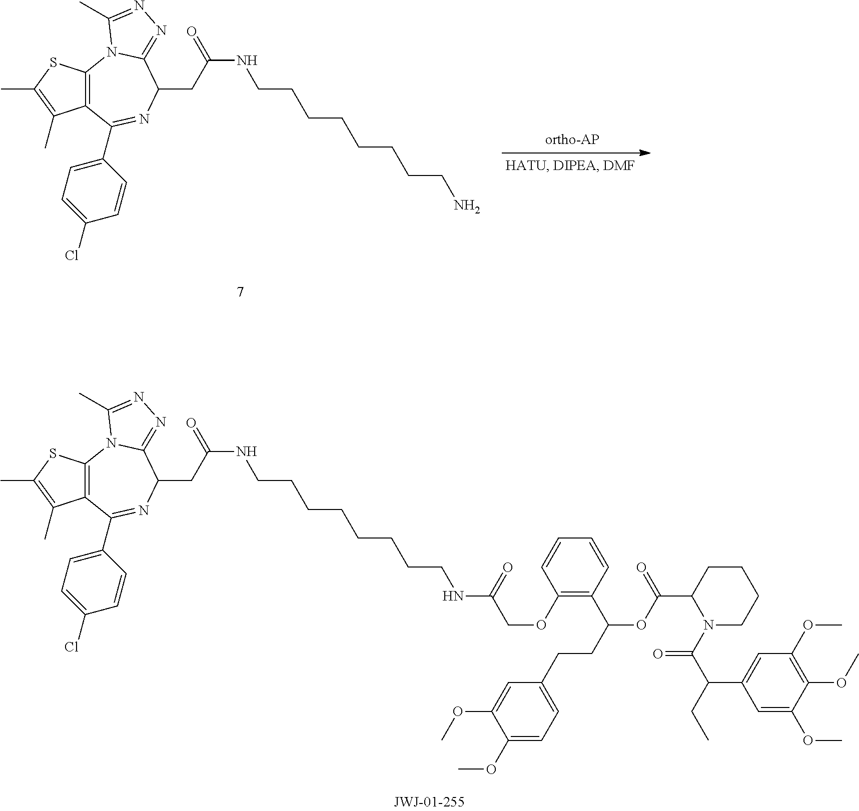

Compound 7 (24 mg, 0.04 mmol), ortho-AP (23 mg, 0.04 mmol), HATU (17 mg, 0.05 mmol), DIPEA (30 μL, 0.19 mmol) were dissolved in DMF (2 mL) and stirred at rt for 16 h. The reaction mixture was filtered and purified by HPLC to afford the title compound as a while solid (3.1 mg, 6.3%) with 99% purity. ¹H NMR (600 MHz, DMSO-d₆) δ 8.15 (t, J=5.6 Hz, 1H), 7.52-7.45 (m, 2H), 7.42 (d, J=8.2 Hz, 2H), 6.89-6.72 (m, 4H), 6.66-6.58 (m, 1H), 6.55 (s, 1H), 6.03 (dd, J=8.3, 4.9 Hz, 1H), 5.32 (dd, J=6.0, 2.5 Hz, 1H), 4.62-4.39 (m, 4H), 3.73 (d, J=4.2 Hz, 2H), 3.71 (s, 3H), 3.69 (s, 3H), 3.55 (d, J=9.3 Hz, 8H), 3.28-3.15 (m, 2H), 3.15-3.01 (m, 5H), 2.59 (s, 5H), 2.40 (d, J=3.6 Hz, 4H), 2.22-2.09 (m, 1H), 2.06-1.83 (m, 2H), 1.65-1.53 (m, 7H), 1.37 (ddt, J=39.8, 13.6, 6.8 Hz, 6H), 1.28-1.13 (m, 12H), 0.76 (dt, J=43.4, 7.3 Hz, 3H). MS (ESI) m/z 1203.6 (M+H)⁺.

### j. tert-Butyl acetyl(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)carbamate (8)

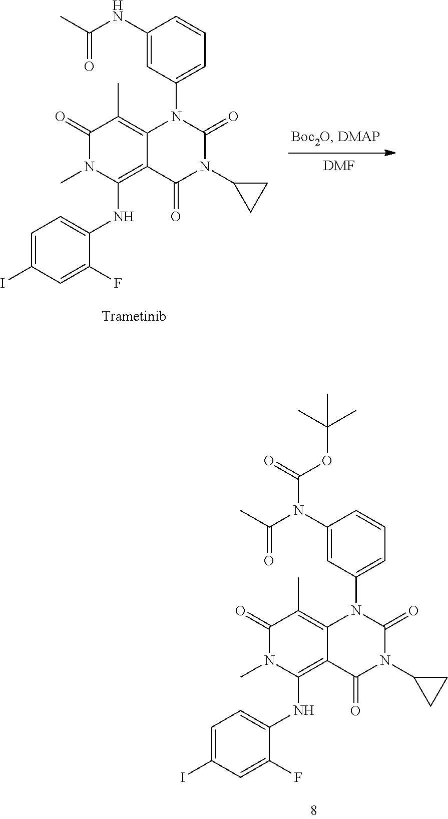

According to a reported procedure,¹ to a solution of trametinib (125 mg, 0.20 mmol), 4-(dimethylamino)pyridine (DMAP; 49.6 mg, 0.41 mmol) and DMF (1.5 mL) in an 8 mL vial, was added a solution of di-tert-butyl dicarbonate (Boc₂O; 133 mg, 0.61 mmol) and DMF (1.5 mL) dropwise over 1 min. The vial was sealed under nitrogen and the solution was stirred for 1 h. The solution was transferred to a 50 mL flask and concentrated to dryness to afford the title compound 8 without further purification, MS (ESI) m/z 716.2 (M+H)⁺.

### k. tert-Butyl (3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)carbamate (9)

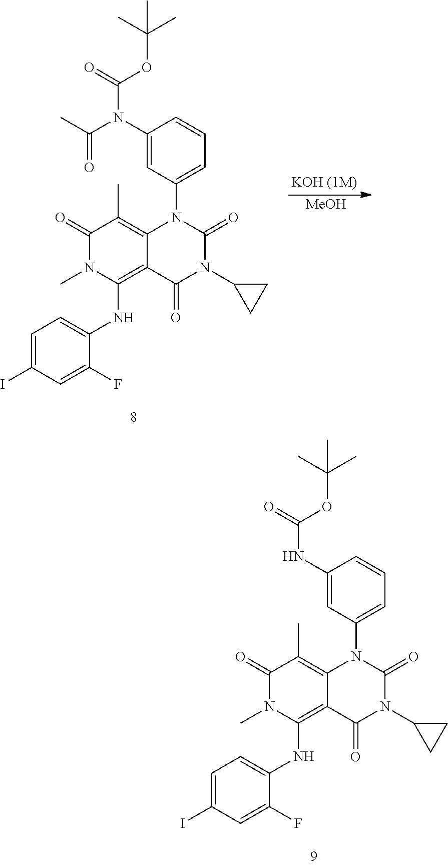 [SMILES: "MeOH" ; CC(=O)N(C(=O)OC(C)(C)C)c1cccc(-n2c(=O)n(C3CC3)c(=O)c3c(Nc4ccc(I)cc4F)n(C)c(=O)c(C)c32)c1 ; Cc1c(=O)n(C)c(Nc2ccc(I)cc2F)c2c(=O)n(C3CC3)c(=O)n(-c3cccc(NC(=O)OC(C)(C)C)c3)c12]

The crude solid 8 was dissolved in MeOH (1.5 mL) and then an aqueous solution of KOH (1.0 mL, 1.0 μM solution, 1.0 mmol) was added. The solution was stirred for 4 h, then diluted with brine (30 mL) and extracted with DCM (3×15 mL). The organic extracts were pooled, dried over magnesium sulfate, filtered, and concentrated to dryness, which yielded the title compound 9 without further purification, MS (ESI) m/z 674.3 (M+H)⁺.

### l. 1-(3-Aminophenyl)-3-cyclopropyl-5-((2-fluoro-4-iodophenyl)amino)-6,8-dimethylpyrido[4,3-d]pyrimidine-2,4,7(1H,3H,6H)-trione (10)

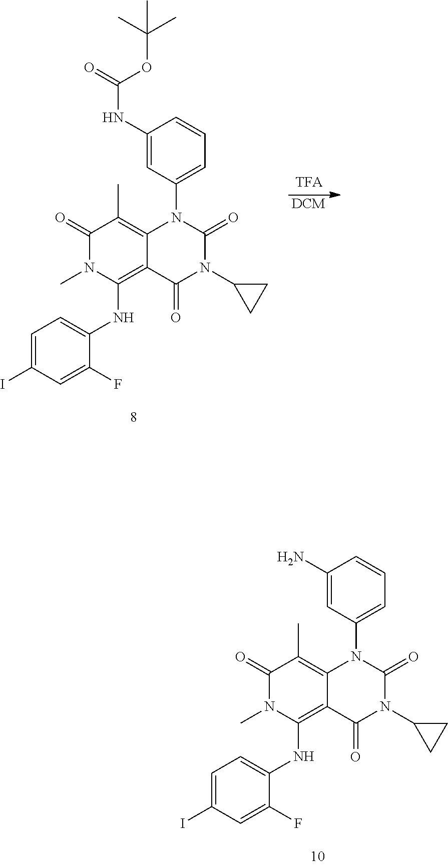

The resulting solid 9 was dissolved in TFA (3.0 mL, 39 mmol). The solution was stirred for 30 min and then concentrated to dryness from toluene (3×3 mL). The remaining material was partitioned between DCM (25 mL) and saturated NaHCO₃ solution (25 mL), and transferred to a separatory funnel. The layers were separated, and the aqueous phase was further extracted with DCM (25 mL). The organic extracts were dried over magnesium sulfate, filtered, and concentrated to dryness. The residue was purified by silica gel chromatography to yield 95 mg (81% over 3 steps) of the title compound 10 as an off-white solid. ¹H NMR (500 MHz, DMSO-d₆) δ 11.07 (s, 1H), 7.78 (dd, J=10.3, 1.9 Hz, 1H), 7.56-7.52 (m, 1H), 7.05 (t, J=7.9 Hz, 1H), 6.90 (t, J=8.6 Hz, 1H), 6.59-6.52 (m, 2H), 6.49-6.44 (m, 1H), 5.26 (s, 2H), 3.07 (s, 3H), 2.61 (tt, J=7.2, 4.0 Hz, 1H), 1.35 (s, 3H), 1.00-0.85 (m, 2H), 0.70-0.59 (m, 2H). MS (ESI) m/z 574.1 (M+H)⁺.

### m. tert-Butyl (2-(2-(3-((3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)amino)-3-oxopropoxy)ethoxy)ethyl)carbamate (11)

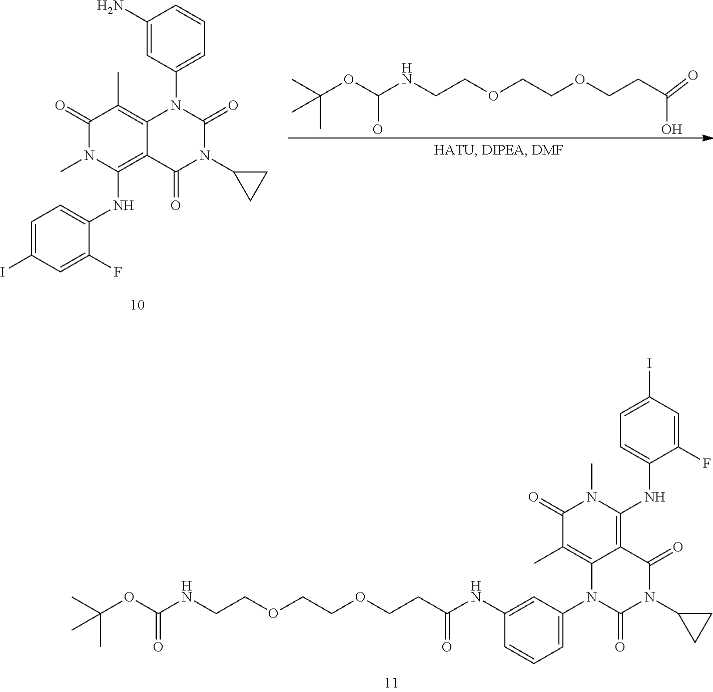 [SMILES: Cc1c(=O)n(C)c(Nc2ccc(I)cc2F)c2c(=O)n(C3CC3)c(=O)n(-c3cccc(N)c3)c12 ; CC(C)(C)OC(O)NCCOCCOCCC(=O)O ; Cc1c(=O)n(C)c(Nc2ccc(I)cc2F)c2c(=O)n(C3CC3)c(=O)n(-c3cccc(NC(=O)CCOCCOCCNC(=O)OC(C)(C)C)c3)c12]

The resulting solid 10 (95 mg, 17 mmol), HATU (76 mg, 20 mmol), DIPEA (50 uL, 26 mmol), and 2,2-dimethyl-4-oxo-3,8,11-trioxa-5-azatetradecan-14-oic acid (51 mg, 18 mmol) were charged into a 8 mL vial. After adding 2 mL DMF, the solution was stirred for 2 hours. Upon the completion of the coupling reaction indicated by LCMS, the material was partitioned between DCM (25 mL) and brine (25 mL), and transferred to a separatory funnel. The layers were separated, and the aqueous phase was further extracted with DCM (25 mL). The organic extracts were dried over magnesium sulfate, filtered, and concentrated to dryness. The residue was purified by silica gel chromatography to yield 112 mg (81O %) of the title compound 11. ¹H NMR (500 MHz, Methanol-d₄) δ 7.71 (t, J=2.1 Hz, 1H), 7.67-7.63 (m, 1H), 7.58-7.50 (m, 2H), 7.38 (t, J=8.1 Hz, 1H), 7.12-7.06 (m, 1H), 6.84 (t, J=8.5 Hz, 1H), 6.53 (s, 1H), 3.80 (t, J=6.1 Hz, 2H), 3.71 (p, J=6.6 Hz, 2H), 3.62-3.60 (m, 2H), 3.60-3.57 (m, 2H), 3.47 (t, J=5.6 Hz, 2H), 3.21 (q, J=7.4 Hz, 2H), 3.18 (s, 3H), 3.16 (t, J=5.5 Hz, 2H), 2.69 (tt, J=7.3, 3.9 Hz, 1H), 2.61 (t, J=6.0 Hz, 2H), 1.40 (s, 10H), 1.03 (dd, J=7.0, 1.5 Hz, 2H), 0.76-0.70 (m, 2H). MS (ESI) m/z 833.4 (M+H)⁺.

### n. 3-(2-(2-Aminoethoxy)ethoxy)-N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)propanamide (12)

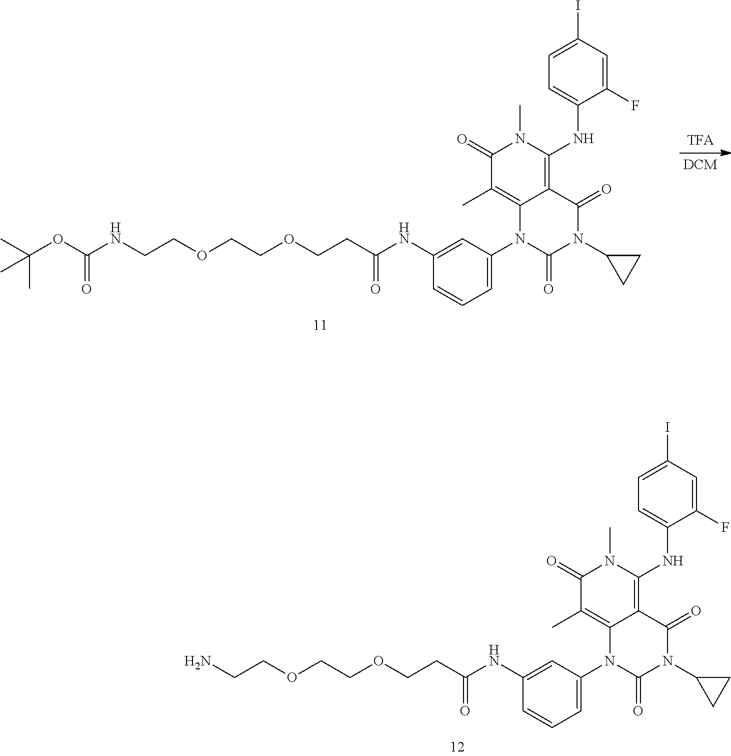 [SMILES: Cc1c(=O)n(C)c(Nc2ccc(I)cc2F)c2c(=O)n(C3CC3)c(=O)n(-c3cccc(NC(=O)CCOCCOCCNC(=O)OC(C)(C)C)c3)c12 ; Cc1c(=O)n(C)c(Nc2ccc(I)cc2F)c2c(=O)n(C3CC3)c(=O)n(-c3cccc(NC(=O)CCOCCOCCN)c3)c12]

Compound 11 (60 mg, 0.07 mmol) was dissolved in 2 mL DCM and 1 mL TFA, and stirred at rt for 2 h. The reaction mixture was concentrated in vacuo and used without further purification (quant.). MS (ESI) m/z 733.2 (M+H)⁺.

### o. (R)-1-(2-(2-((2-(2-(3-((3-(3-Cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)amino)-3-oxopropoxy)ethoxy)ethyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (JWJ-01-280)

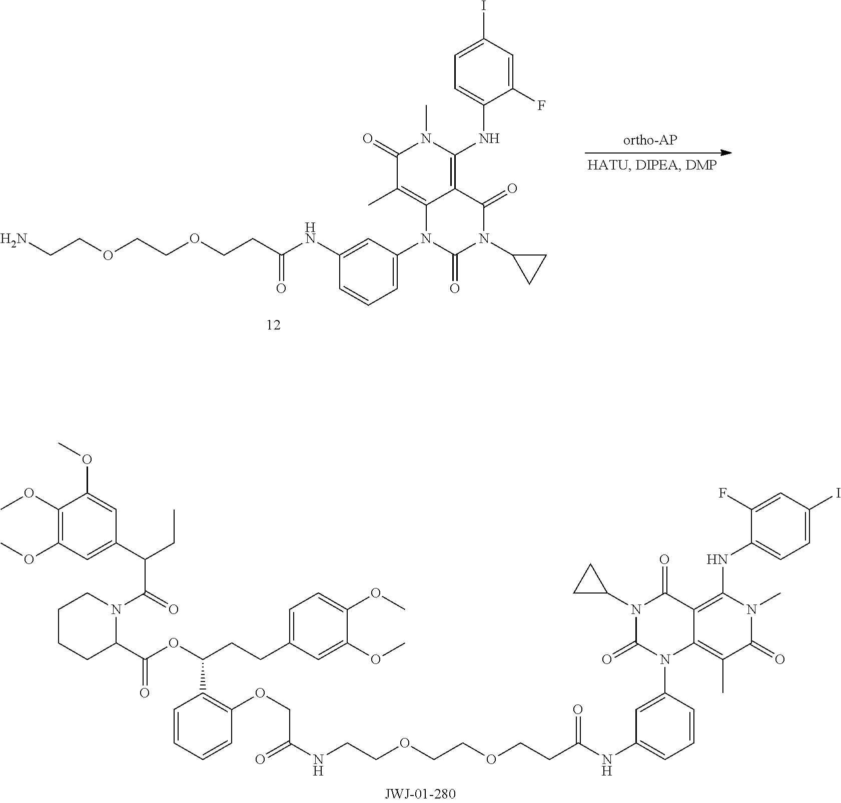 [SMILES: Cc1c(=O)n(C)c(Nc2ccc(I)cc2F)c2c(=O)n(C3CC3)c(=O)n(-c3cccc(NC(=O)CCOCCOCCN)c3)c12 ; CCC(C(=O)N1CCCCC1C(=O)O[C@H](CCc1ccc(OC)c(OC)c1)c1ccccc1OCC(=O)NCCOCCOCCC(=O)Nc1cccc(-n2c(=O)n(C3CC3)c(=O)c3c(Nc4ccc(I)cc4F)n(C)c(=O)c(C)c32)c1)c1cc(OC)c(OC)c(OC)c1]

Compound 12 (42 mg, 0.06 mmol), ortho-AP (20 mg, 0.03 mmol), HATU (13 mg, 0.04 mmol), DIPEA (25 μL, 0.14 mmol) were dissolved in DMF (2 mL) and stirred at rt for 16 h. The reaction mixture was filtered and purified by HPLC to afford the title compound as a while solid (13 mg, 29%) with 98% purity. ¹H NMR (500 MHz, DMSO-d₆) δ 11.07 (s, 1H), 10.11 (s, 1H), 7.84 (t, J=5.7 Hz, 1H), 7.78 (dd, J=10.2, 1.9 Hz, 1H), 7.64 (t, J=2.1 Hz, 1H), 7.61-7.51 (m, 2H), 7.39-7.32 (m, 1H), 7.19 (ddd, J=8.6, 6.7, 2.4 Hz, 1H), 7.02 (dd, J=8.2, 6.0 Hz, 1H), 6.94-6.82 (m, 2H), 6.82-6.76 (m, 3H), 6.73 (d, J=2.0 Hz, 1H), 6.66-6.60 (m, 1H), 6.55 (s, 1H), 6.02 (dd, J=8.3, 4.8 Hz, 1H), 5.34-5.30 (m, 1H), 4.54-4.46 (m, 2H), 3.86 (t, J=7.2 Hz, 2H), 3.74 (s, 2H), 3.70 (s, 3H), 3.68 (s, 3H), 3.66 (t, J=6.4 Hz, 2H), 3.63 (s, 1H), 3.55 (s, 4H), 3.53 (s, 2H), 3.50-3.42 (m, 4H), 3.39 (t, J=5.9 Hz, 2H), 3.30-3.20 (m, 2H), 3.06 (s, 3H), 2.65-2.57 (m, 2H), 2.57-2.52 (m, 3H), 2.44-2.35 (m, 1H), 2.15 (d, J=13.0 Hz, 1H), 2.08-1.83 (m, 3H), 1.66-1.47 (m, 4H), 1.42-1.06 (m, 6H), 0.94 (q, J=6.8 Hz, 2H), 0.76 (dt, J=37.7, 7.3 Hz, 3H), 0.66 (dd, J=7.0, 4.0 Hz, 2H). MS (ESI) m/z 1043.3 (M+H)⁺ one fragment.

### Proposed Fragmentation Pattern

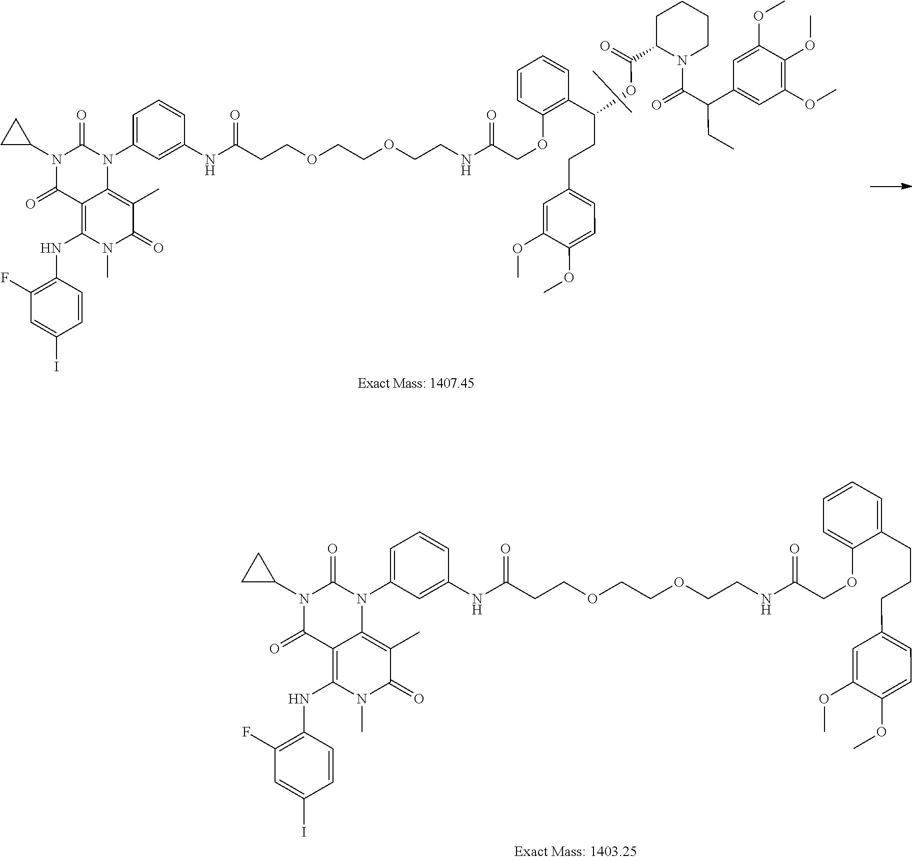

v. General Procedure of HATU Coupling

Corresponding protein-of-interest binder with varying Boc protected linker (1 equiv) was treated with a 1:1 (v/v) mixture of DCM/TFA (5 mL/mmol) for 30 min. Upon the completion of the removal of the Boc group indicated by UPLC, excess TFA and DCM were evaporated under reduced pressure. After putting on the high vacuum for additional 2 hours to fully remove TFA, the residue was dissolved in DMF (1 mL). HATU (1.5 equiv), DIPEA (2 equiv), and the ortho-AP acid (1 equiv) were added to the solution and stirred ovemight. Upon the completion of the HATU coupling indicated by UPLC, the mixture was diluted with DMSO to 5 mL and filtered through a 13 mm syringe filter (0.45 um, PTFE). The resulting filtrate was loaded and purified via the preparative HPLC, which yielded the desired compound as a TFA salt.

### a. tert-Butyl acetyl(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)carbamate

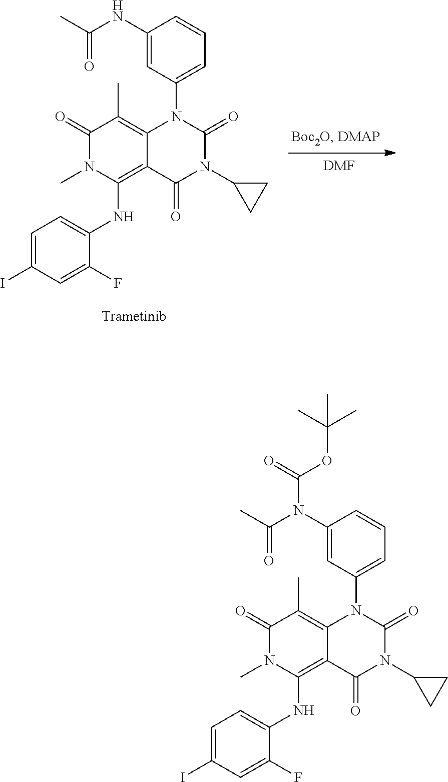

According to a reported procedure,¹ to a solution of trametinib (125 mg, 0.20 mmol), 4-(dimethylamino)pyridine (DMAP; 49.6 mg, 0.41 mmol) and DMF (1.5 mL) in an 8 mL vial, was added a solution of di-tert-butyl dicarbonate (Boc₂O; 133 mg, 0.61 mmol) and DMF (1.5 mL) dropwise over 1 min. The vial was sealed under nitrogen and the solution was stirred for 1 h. The solution was transferred to a 50 mL flask and concentrated to dryness to afford the title compound without further purification.

### b. tert-Butyl (3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)carbamate

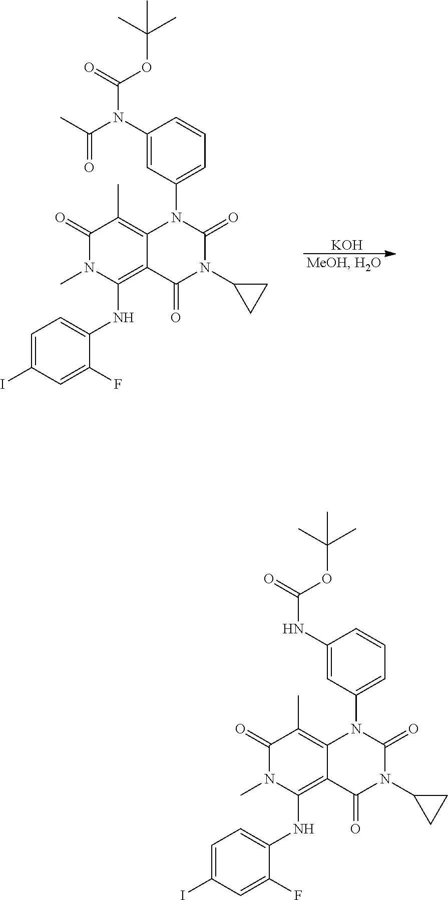

The crude solid was dissolved in MeOH (1.5 mL) and then an aqueous solution of KOH (1.0 mL, 1.0 μM solution, 1.0 mmol) was added. The solution was stirred for 4 h, then diluted with brine (30 mL) and extracted with DCM (3×15 mL). The organic extracts were pooled, dried over magnesium sulfate, filtered, and concentrated to dryness, which yielded the title compound without further purification.

### c. 1-(3-Aminophenyl)-3-cyclopropyl-5-((2-fluoro-4-iodophenyl)amino)-6,8-dimethylpyrido[4,3-d]pyrimidine-2,4,7(1H,3H,6H)-trione

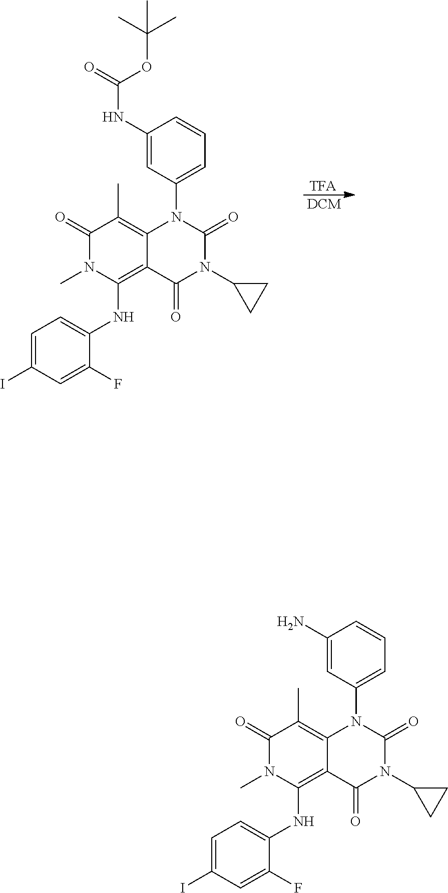

The resulting solid was dissolved in trifluoroacetic acid (TFA, 3.0 mL, 39 mmol). The solution was stirred for 30 min and then concentrated to dryness from toluene (3×3 mL). The remaining material was partitioned between DCM (25 mL) and saturated NaHCO₃ solution (25 mL), and transferred to a separatory funnel. The layers were separated, and the aqueous phase was further extracted with DCM (25 mL). The organic extracts were dried over magnesium sulfate, filtered, and concentrated to dryness. The residue was purified by silica gel chromatography to yield 95 mg (81% over 3 steps) of the title compound as an off-white solid.

¹H NMR (500 MHz, DMSO-d₆) δ 11.07 (s, 1H), 7.78 (dd, J=10.3, 1.9 Hz, 1H), 7.56-7.52 (m, 1H), 7.05 (t, J=7.9 Hz, 1H), 6.90 (t, J=8.6 Hz, 1H), 6.59-6.52 (m, 2H), 6.49-6.44 (m, 1H), 5.26 (s, 2H), 3.07 (s, 3H), 2.61 (tt, J=7.2, 4.0 Hz, 1H), 1.35 (s, 3H), 1.00-0.85 (m, 2H), 0.70-0.59 (m, 2H).

### d. tert-Butyl (2-(2-(3-((3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)amino)-3-oxopropoxy)ethoxy)ethyl)carbamate

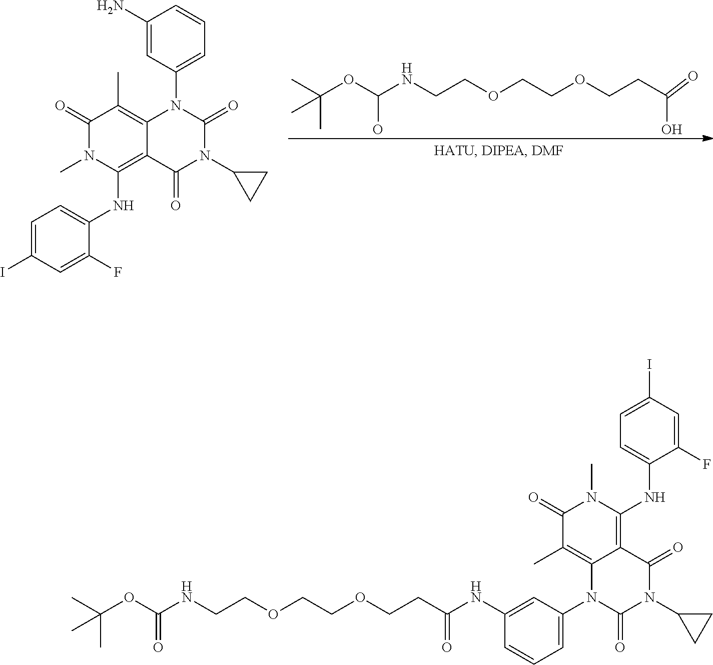 [SMILES: Cc1c(=O)n(C)c(Nc2ccc(I)cc2F)c2c(=O)n(C3CC3)c(=O)n(-c3cccc(N)c3)c12 ; CC(C)(C)OC(O)NCCOCCOCCC(=O)O ; Cc1c(=O)n(C)c(Nc2ccc(I)cc2F)c2c(=O)n(C3CC3)c(=O)n(-c3cccc(NC(=O)CCOCCOCCNC(=O)OC(C)(C)C)c3)c12]

The resulting solid (95 mg, 17 mmol), HATU (76 mg, 20 mmol), DIPEA (50 uL, 26 mmol), and 2,2-dimethyl-4-oxo-3,8,11-trioxa-5-azatetradecan-14-oic acid (51 mg, 18 mmol) were charged into a 8 mL vial. After adding 2 mL DMF, the solution was stirred for 2 hours. Upon the completion of the coupling reaction indicated by LCMS, the material was partitioned between DCM (25 mL) and brine (25 mL), and transferred to a separatory funnel. The layers were separated, and the aqueous phase was further extracted with DCM (25 mL). The organic extracts were dried over magnesium sulfate, filtered, and concentrated to dryness. The residue was purified by silica gel chromatography to yield 112 mg (81%) of the title compound. ¹H NMR (500 MHz, Methanol-d₄) δ 7.71 (t, J=2.1 Hz, 1H), 7.67-7.63 (m, 1H), 7.58-7.50 (m, 2H), 7.38 (t, J=8.1 Hz, 1H), 7.12-7.06 (m, 1H), 6.84 (t, J=8.5 Hz, 1H), 6.53 (s, 1H), 3.80 (t, J=6.1 Hz, 2H), 3.71 (p, J=6.6 Hz, 2H), 3.62-3.60 (m, 2H), 3.60-3.57 (m, 2H), 3.47 (t, J=5.6 Hz, 2H), 3.21 (q, J=7.4 Hz, 2H), 3.18 (s, 3H), 3.16 (t, J=5.5 Hz, 2H), 2.69 (tt, J=7.3, 3.9 Hz, 1H), 2.61 (t, J=6.0 Hz, 2H), 1.40 (s, 10H), 1.03 (dd, J=7.0, 1.5 Hz, 2H), 0.76-0.70 (m, 2H).

### e. (R)-1-(2-(2-((2-(2-(3-((3-(3-Cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)amino)-3-oxopropoxy)ethoxy)ethyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (JWJ-01-280)

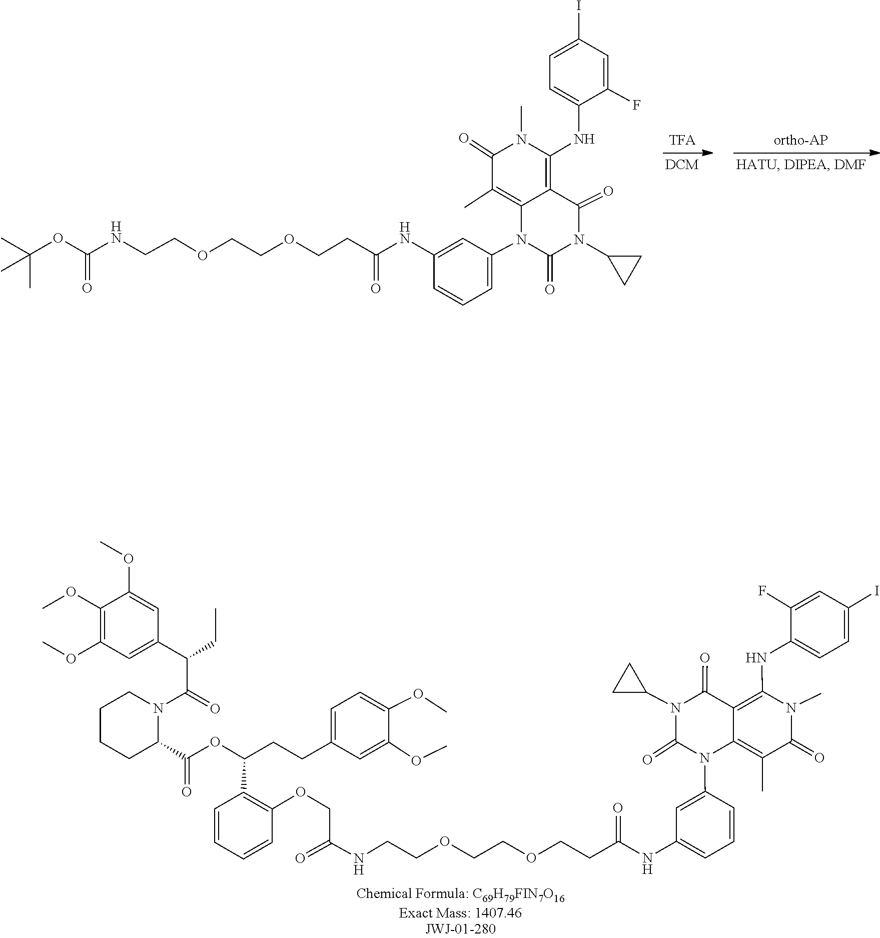 [SMILES: Cc1c(=O)n(C)c(Nc2ccc(I)cc2F)c2c(=O)n(C3CC3)c(=O)n(-c3cccc(NC(=O)CCOCCOCCNC(=O)OC(C)(C)C)c3)c12 ; CC[C@H](C(=O)N1CCCC[C@H]1C(=O)O[C@H](CCc1ccc(OC)c(OC)c1)c1ccccc1OCC(=O)NCCOCCOCCC(=O)Nc1cccc(-n2c(=O)n(C3CC3)c(=O)c3c(Nc4ccc(I)cc4F)n(C)c(=O)c(C)c32)c1)c1cc(OC)c(OC)c(OC)c1]

Title compound was generated via general procedure of HATU coupling using resulting solid (42 mg, 0.058 mmol) and ortho-AP acid (20 mg, 0.029 mmol), which yielded a while solid (13 mg, 29%) with 98% purity. ¹H NMR (500 MHz, DMSO-d₆) δ 11.07 (s, 1H), 10.11 (s, 1H), 7.84 (t, J=5.7 Hz, 1H), 7.78 (dd, J=10.2, 1.9 Hz, 1H), 7.64 (t, J=2.1 Hz, 1H), 7.61-7.51 (m, 2H), 7.39-7.32 (m, 1H), 7.19 (ddd, J=8.6, 6.7, 2.4 Hz, 1H), 7.02 (dd, J=8.2, 6.0 Hz, 1H), 6.94-6.82 (m, 2H), 6.82-6.76 (m, 3H), 6.73 (d, J=2.0 Hz, 1H), 6.66-6.60 (m, 1H), 6.55 (s, 1H), 6.02 (dd, J=8.3, 4.8 Hz, 1H), 5.34-5.30 (m, 1H), 4.54-4.46 (m, 2H), 3.86 (t, J=7.2 Hz, 2H), 3.74 (s, 2H), 3.70 (s, 3H), 3.68 (s, 3H), 3.66 (t, J=6.4 Hz, 2H), 3.63 (s, 1H), 3.55 (s, 4H), 3.53 (s, 2H), 3.50-3.42 (m, 4H), 3.39 (t, J=5.9 Hz, 2H), 3.30-3.20 (m, 2H), 3.06 (s, 3H), 2.65-2.57 (m, 2H), 2.57-2.52 (m, 3H), 2.44-2.35 (m, 1H), 2.15 (d, J=13.0 Hz, 1H), 2.08-1.83 (m, 3H), 1.66-1.47 (m, 4H), 1.42-1.06 (m, 6H), 0.94 (q, J=6.8 Hz, 2H), 0.76 (dt, J=37.7, 7.3 Hz, 3H), 0.66 (dd, J=7.0, 4.0 Hz, 2H). MS (ESI) m/z 1043.3 (M+H)⁺ major fragment.

### f. tert-Butyl (S)-(8-(2-(4-(4-chlorophenyl)-2,3,9-trimethyl-6H-thieno[3,2-f][1,2,4]triazolo[4,3-a][1,4]diazepin-6-yl)acetamido)octyl)carbamate

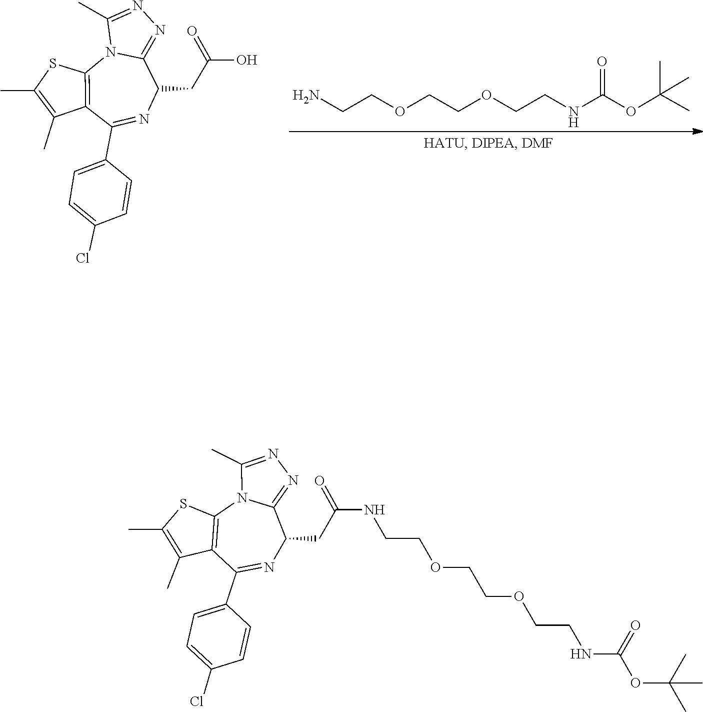

(+)-JQ1 carboxylic acid (25 mg, 0.062 mmol), HATU (28 mg, 0.075 mmol), DIPEA (33 uL, 0.19 mmol), and tert-butyl (2-(2-(2-aminoethoxy)ethoxy)ethyl)carbamate (17 mg, 0.069 mmol) were charged into a 4 mL vial. After adding 1 mL DMF, the solution was stirred for 2 hours. Upon the completion of the coupling reaction indicated by LCMS, the material was partitioned between DCM (15 mL) and brine (15 mL), and transferred to a separatory funnel. The layers were separated, and the aqueous phase was further extracted with DCM (15 mL). The organic extracts were dried over magnesium sulfate, filtered, and concentrated to dryness. The residue was purified by silica gel chromatography to yield 37 mg (95%) of the title compound.

¹H NMR (500 MHz, Methanol-d₄) δ 7.44 (d, J=8.3 Hz, 2H), 7.39 (d, J=8.7 Hz, 2H), 4.61 (dd, J=8.9, 5.3 Hz, 1H), 3.61 (ddd, J=11.9, 6.9, 3.5 Hz, 6H), 3.50 (t, J=5.7 Hz, 2H), 3.46-3.42 (m, 2H), 3.31 (d, J=5.2 Hz, 1H), 3.27 (s, 2H), 3.20 (t, J=5.6 Hz, 2H), 2.68 (s, 3H), 2.43 (s, 3H), 1.68 (s, 3H), 1.40 (s, 9H), 1.29 (dd, J=12.4, 6.3 Hz, 1H).

### g. (R)-1-(2-((14-((S)-4-(4-Chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-3-(3,4-dimethoxyphenyl)propyl (S)-1-((S)-2-(3,4,5-trimethoxyphenyl)butanoyl)piperidine-2-carboxylate (JWJ-01-236)

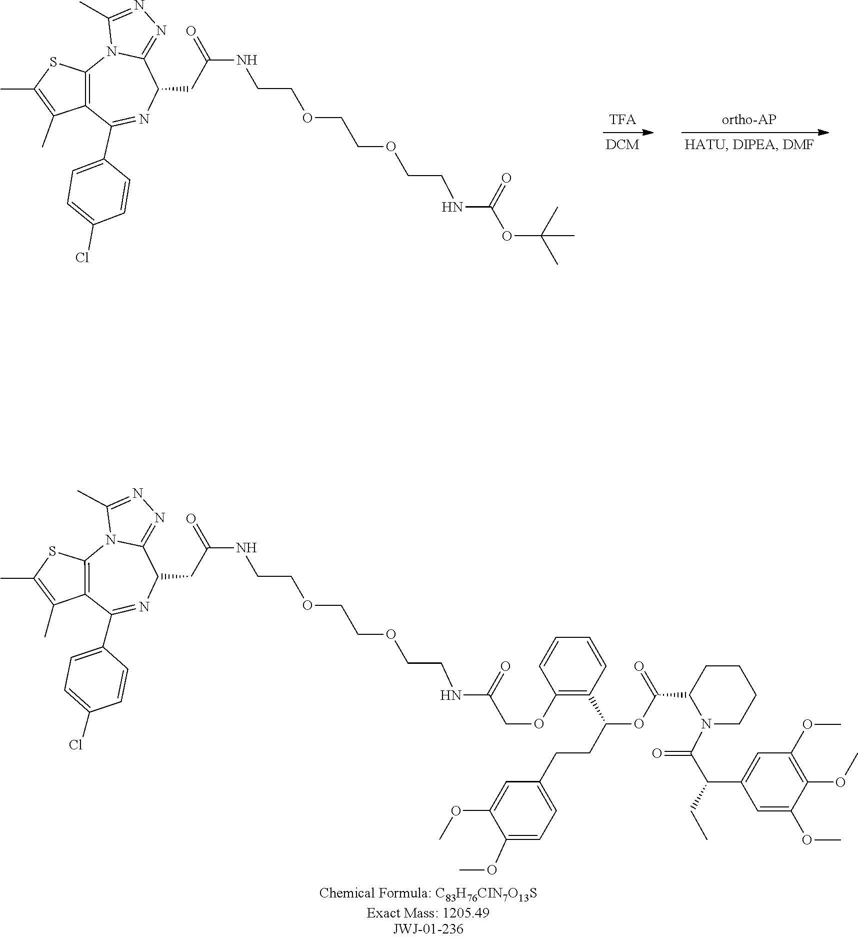

Title compound was generated via general procedure of HATU coupling using resulting solid (37 mg, 0.047 mmol) and ortho-AP acid (36 mg, 0.052 mmol), which yielded a while solid (1.6 mg, 2.6%) with 95% purity. ¹H NMR (500 MHz, DMSO-d₆) δ 8.26 (t, J=5.6 Hz, 1H), 7.85 (t, J=5.8 Hz, 1H), 7.48 (d, J=8.7 Hz, 2H), 7.42 (d, J=8.3 Hz, 2H), 7.19 (ddd, J=8.6, 6.6, 2.4 Hz, 1H), 6.88-6.72 (m, 4H), 6.65-6.60 (m, 1H), 6.55 (s, 1H), 6.02 (dd, J=8.3, 4.7 Hz, 1H), 5.36-5.29 (m, 2H), 4.60-4.49 (m, 3H), 4.05 (d, J=13.4 Hz, 1H), 3.87 (t, J=7.2 Hz, 1H), 3.74 (s, 1H), 3.71 (s, 3H), 3.69 (s, 3H), 3.64 (d, J=12.5 Hz, 1H), 3.55 (s, 4H), 3.54 (s, 2H), 3.50 (t, J=5.7 Hz, 5H), 3.48-3.38 (m, 5H), 3.32-3.18 (m, 4H), 2.60 (s, 4H), 2.54 (s, 2H), 2.40 (s, 4H), 2.16 (d, J=13.0 Hz, 1H), 2.04-1.84 (m, 3H), 1.61 (s, 7H), 1.42-0.90 (m, 3H), 0.76 (dt, J=37.5, 7.3 Hz, 3H). MS (ESI) m/z 1206.6 (M+H)⁺.

### h. tert-Butyl (S)-(8-(2-(4-(4-chlorophenyl)-2,3,9-trimethyl-6H-thieno[3,2-f][1,2,4]triazolo[4,3-a][1,4]diazepin-6-yl)acetamido)octyl)carbamate

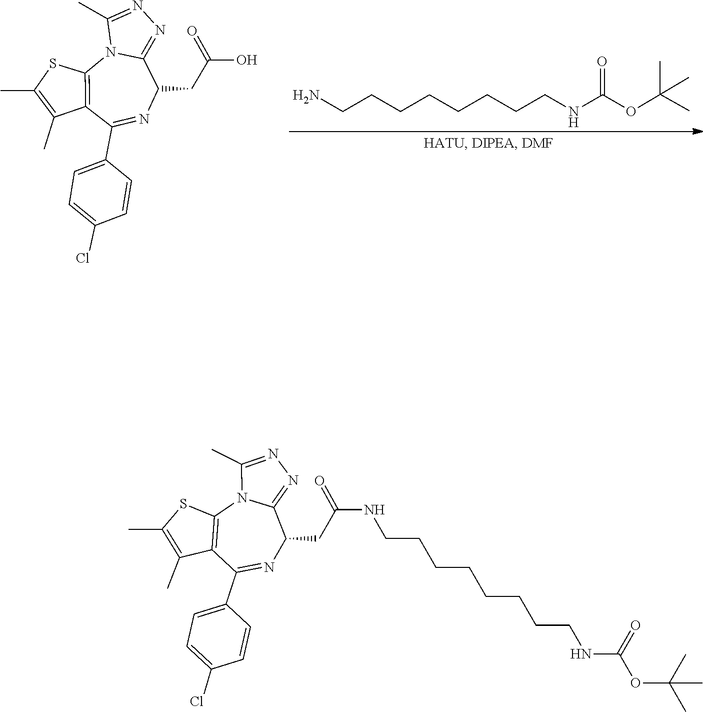

(+)-JQ1 carboxylic acid (15 mg, 0.037 mmol), HATU (14 mg, 0.056 mmol), DIPEA (20 uL, 0.11 mmol), and tert-butyl (8-aminooctyl)carbamate (11 mg, 0.045 mmol) were charged into a 4 mL vial. After adding 1 mL DMF, the solution was stirred for 2 hours. Upon the completion of the coupling reaction indicated by LCMS, the material was partitioned between DCM (15 mL) and brine (15 mL), and transferred to a separatory funnel. The layers were separated, and the aqueous phase was further extracted with DCM (15 mL). The organic extracts were dried over magnesium sulfate, filtered, and concentrated to dryness. The residue was purified by silica gel chromatography to yield 23 mg (99%) of the title compound.

### i. (R)-1-(2-(2-((8-(2-((S)-4-(4-Chlorophenyl)-2,3,9-trimethyl-6H-thieno[3,2-f][1,2,4]triazolo[4,3-a][1,4]diazepin-6-yl)acetamido)octyl)amino)-2-oxoethoxy)phenyl)-3-(3,4-dimethoxyphenyl)propyl (S)-1-((S)-2-(3,5-trimethoxyphenyl)butanoyl)piperidine-2-carboxylate (JWJ-01-255)

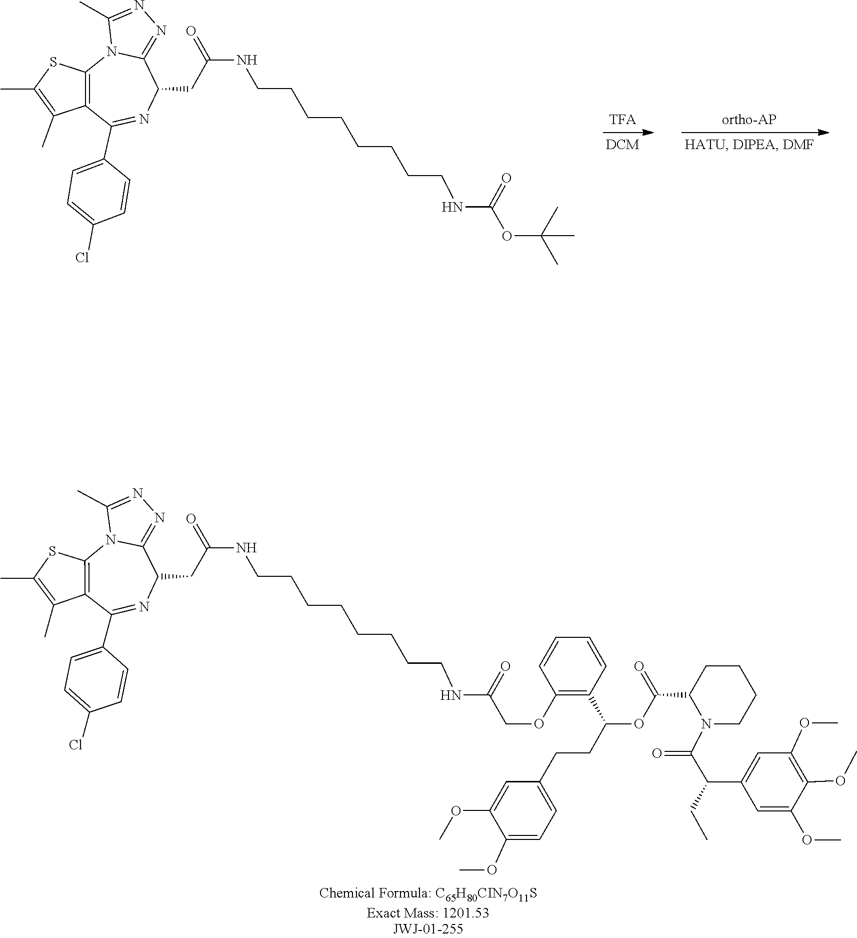

Title compound was generated via general procedure of HATU coupling using resulting solid (23 mg, 0.037 mmol) and ortho-AP acid (23 mg, 0.034 mmol), which yielded a while solid (3.1 mg, 6.3%) with 99% purity. ¹H NMR (600 MHz, DMSO-d₆) δ 8.15 (t, J=5.6 Hz, 1H), 7.52-7.45 (m, 2H), 7.42 (d, J=8.2 Hz, 2H), 6.89-6.72 (m, 4H), 6.66-6.58 (m, 1H), 6.55 (s, 1H), 6.03 (dd, J=8.3, 4.9 Hz, 1H), 5.32 (dd, J=6.0, 2.5 Hz, 1H), 4.62-4.39 (m, 4H), 3.73 (d, J=4.2 Hz, 2H), 3.71 (s, 3H), 3.69 (s, 3H), 3.55 (d, J=9.3 Hz, 8H), 3.28-3.15 (m, 2H), 3.15-3.01 (m, 5H), 2.59 (s, 5H), 2.40 (d, J=3.6 Hz, 4H), 2.22-2.09 (m, 1H), 2.06-1.83 (m, 2H), 1.65-1.53 (m, 7H), 1.37 (ddt, J=39.8, 13.6, 6.8 Hz, 6H), 1.28-1.13 (m, 12H), 0.76 (dt, J=43.4, 7.3 Hz, 3H). MS (ESI) m/z 1203.6 (M+H)⁺.

Those skilled in the art will recognize, or be able to ascertain using no more than routine experimentation, many equivalents to the specific embodiments of the method and compositions described herein. Such equivalents are intended to be encompassed by the following claims.

### REFERENCES

1 Khan, Z. M. et al. Structural basis for the action of the drug trametinib at KSR-bound MEK. Nature 588, 509-514 (2020). https://doi.org-10.1038/s41586-020-2760-4
2 Ferguson, F. M. & Gray, N. S. Kinase inhibitors: the road ahead. Nat Rev Drug Discov 17, 353-377 (2018). https://doi.org:10.1038/nrd.2018.21
3 Ito, T. et al. Identification of a primary target of thalidomide teratogenicity. Science 327, 1345-1350 (2010). https://doi.org:10.1126/science.1177319
4 Kronke, J. et al. Lenalidomide causes selective degradation of IKZF1 and IKZF3 in multiple myeloma cells. Science 343, 301-305 (2014). https://doi.org:10.1126/science.1244851
5 Lu, G. et al. The myeloma drug lenalidomide promotes the cereblon-dependent destruction of Ikaros proteins. Science 343, 305-309 (2014). https://doi.org:10.1126/science.1244917
6 Holderfield, M., Nagel, T. E. & Stuart, D. D. Mechanism and consequences of RAF kinase activation by small-molecule inhibitors. Br J Cancer 111, 640-645 (2014). https://doi.org.10.1038/bjc.2014.139
7 Shu, S. et al. Response and resistance to BET bromodomain inhibitors in triple-negative breast cancer. Nature 529, 413-417 (2016). https://doi.org:10.1038/nature16508
8 Iniguez, A. B. et al. Resistance to Epigenetic-Targeted Therapy Engenders Tumor Cell Vulnerabilities Associated with Enhancer Remodeling. Cancer Cell 34, 922-938 e927 (2018). https://doi.org:10.1016/1 ccell.2018.11.005
9 West, A. V. & Woo, C. M. Photoaffinity Labeling Chemistries Used to Map Biomolecular Interactions. Israel Journal of Chemistry, e202200081 (2022).
10 Trowbridge, A. D. et al. Small molecule photocatalysis enables drug target identification via energy transfer. Proc Natl Acad Sci USA 119, e2208077119 (2022). https://doi.org.10.1073/pnas.2208077119
11 Richards, A. L., Eckhardt, M. & Krogan, N. J. Mass spectrometry-based protein-protein interaction networks for the study of human diseases. Mol Syst Biol 17, e8792 (2021). https://doi.org:10.15252/msb.20188792
12 May, D. G., Scott, K. L., Campos, A. R. & Roux, K. J. Comparative Application of BioID and TurboID for Protein-Proximity Blotinylation. Cells 9 (2020). https://doi.org:10.3390/cells9051070
13 Qin, W., Cho, K. F., Cavanagh, P. E. & Ting, A. Y. Deciphering molecular interactions by proximity labeling. Nat Methods 18, 133-143 (2021). https.//doi.org:10.1038/s41592-020-01010-5
14 Filippakopoulos, P. et al. Selective inhibition of BET bromodomains. Nature 468, 1067-1073 (2010). https://doi.org:10.1038/nature09504
15 Nabet, B. et al. Rapid and direct control of target protein levels with VHL-recruiting dTAG molecules. Nat Commun 11, 4687 (2020). https://doi.org:10.1038/s41467-020-18377-w
16 Lambert, J. P. et al. Interactome Rewiring Following Pharmacological Targeting of BET Bromodomains. Mol Cell 73, 621-638 e617 (2019). https://doi.org:10.1016/j.molcel.2018.11.006
17 Ashbumer, M. et al. Gene ontology: tool for the unification of biology. The Gene Ontology Consortium. Nat Genet 25, 25-29 (2000). https.//doi.org.10.1038/75556
18 Gene Ontology, C. The Gene Ontology resource: enriching a GOld mine. Nucleic Acids Res 49, D325-D334 (2021). https://doi.org:10.1093/nar/gkaa1113
19 Schwalm, M. P. & Knapp, S. BET bromodomain inhibitors. Cuff Opin Chem Biol 68, 102148 (2022). https://doi.org:10.1016/i.cbpa.2022.102148
20 Domostegui, A., Nieto-Barrado, L., Perez-Lopez, C. & Mayor-Ruiz, C. Chasing molecular glue degraders: screening approaches. Chem Soc Rev 51, 5498-5517 (2022). https.//doi.org:10.1039/d2cs001970
21 Mayor-Ruiz, C. et al. Publisher Correction: Rational discovery of molecular glue degraders via scalable chemical profiling. Nat Chem Biol 17, 361 (2021). https://doi.org:10.1038/s41589-021-00735-4
22 King, E. A. et al. Chemoproteomics-Enabled Discovery of a Covalent Molecular Glue Degrader Targeting NF-κB. bioRxiv, 2022.2005.2018.492542 (2022). https://doi.org:10.1101/2022.05.18.492542
23 Lazear, M. R. et al. Proteomic discovery of chemical probes that perturb protein complexes in human cells. bioRxiv, 2022.2012.2012.520090 (2022). https://doi.org.10.1101/2022.12.12.520090
24 Cho, K. F. et al. Proximity labeling in mammalian cells with TurboID and split-TurboID. Nat Protoc 15, 3971-3999 (2020). https://doi.org:10.1038/s41596-020-0399-0
25 Venable, J. D. et al. Drug Interaction mapping with proximity dependent enzyme recruiting chimeras. bioRxiv, 2022.2009.2026.509259 (2022). https://doi.org.10.1101/2022.09.26.509259

We claim:

1. A composition comprising a ligandable tag, a linker, and a labeling enzyme, wherein the linker attaches the ligandable tag to the labeling enzyme.

2. The composition of claim 1, wherein the labeling enzyme is an enzyme which can label a protein for biochemical analysis.

3. The composition of claim 2, wherein the labeling enzyme is a protein having ligase activity.

4. The composition of claim 3, wherein the protein having ligase activity is a biotin ligase.

5. The composition of claim 4, wherein the biotin ligase is miniTurboID, TurboID, AirID, splitTurboID, or BirA.

6. The composition of claim 3, wherein the protein having ligase activity is Formlyglycine, sortase, transglutaminase, farnesyltransferase, APEX, or lipoic acid ligase.

7. The composition of any one of claims 1-6, wherein the ligandable tag is a peptide or nucleic acid.

8. The composition of any one of claims 1-7, wherein the ligandable tag is FKBP12^{F36V}.

9. The composition of any one of claims 1-7, wherein the ligandable tag is HaloTAG, SNAp-tag, CLIp-TAG, LigandLink, or cutinase.

10. The composition of any one of claims 1-9, wherein the linker is a flexible linker.

11. The composition of any one of claims 1-10, wherein the linker is 6×(GSS)

12. The composition of any one of claims 1-11, wherein the linker attaches the C-terminal end of the labeling enzyme with the N-terminal end of the ligandable tag.

13. The composition of any one of claims 1-11, wherein the linker attaches the N-terminal end of the labeling enzyme with the N-terminal end of the ligandable tag.

14. The composition of any one of claims 1-11, wherein the linker attaches the C-terminal end of the labeling enzyme with the C-terminal end of the ligandable tag.

15. The composition of any one of claims 1-11, wherein the linker attaches the N-terminal end of the labeling enzyme with the C-terminal end of the ligandable tag.

16. The composition of any one of claims 1-15, further comprising a detection moiety or a purification moiety.

17. The composition of claim 16, wherein the detection moiety or purification moiety is a fluorescent molecule, biotin, streptavidin, c-myc, HA, GST, histidines.

18. A nucleic acid construct capable of encoding the composition of any one of claims 1-17.

19. A nucleic acid construct comprising a nucleic acid sequence capable of encoding a ligandable tag, a nucleic acid sequence capable of encoding a linker, and a nucleic acid sequence capable of encoding a labeling enzyme.

20. The nucleic acid construct of claims 18-19, further comprising a promoter.

21. The nucleic acid construct of claim 20, wherein the promoter is an inducible promoter.

22. The nucleic acid construct of claim 20, wherein the promoter is PGK.

23. The nucleic acid construct of any one of claims 19-22, further comprising a nucleic acid sequence capable of encoding a detection moiety or purification moiety.

24. The nucleic acid construct of claim 23, wherein the detection moiety or purification moiety is a fluorescent molecule, biotin, c-myc, HA, GST, histidines.

25. A vector comprising the nucleic acid construct of claims 18-24.

26. The vector of claim 25, wherein the vector is an expression vector.

27. The vector of claim 26, wherein the expression vector is a viral vector.

28. A composition comprising a ligand conjugated to a molecule of interest.

29. The composition of claim 28, wherein the composition comprises a linker between the ligand and molecule of interest.

30. The composition of claim 29, wherein the linker is a chemical linker.

31. The composition of any one of claims 28-30, wherein the ligand is Ortho-AP1867.

32. The composition of any one of claims 28-31, wherein the ligand is a HaloTag ligand.

33. The composition of claim 32, wherein the HaloTag ligand is a haloalkane.

34. The composition of any one of claims 28-33, wherein the molecule of interest is ribociclib.

35. The composition of any one of claims 28-33, wherein the molecule of interest is (+)-S-JQ1.

36. The composition of any one of claims 28-33, wherein the molecule of interest is trametinib.

37. A composition comprising the structure of

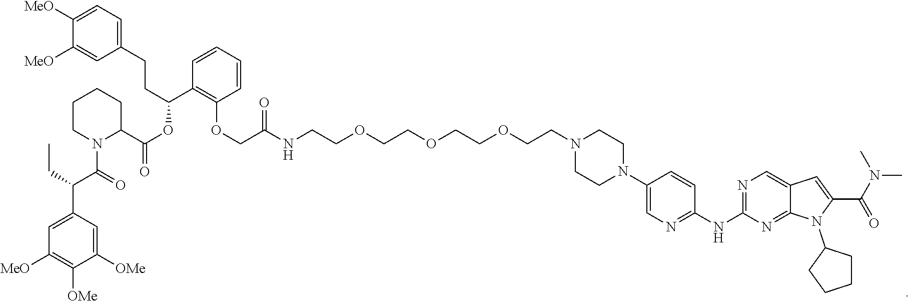 [SMILES: CC[C@H](C(=O)N1CCCCC1C(=O)O[C@H](CCc1ccc(OC)c(OC)c1)c1ccccc1OCC(=O)NCCOCCOCCOCCN1CCN(c2ccc(Nc3ncc4cc(C(=O)N(C)C)n(C5CCCC5)c4n3)nc2)CC1)c1cc(OC)c(OC)c(OC)c1]

38. A composition comprising the structure of

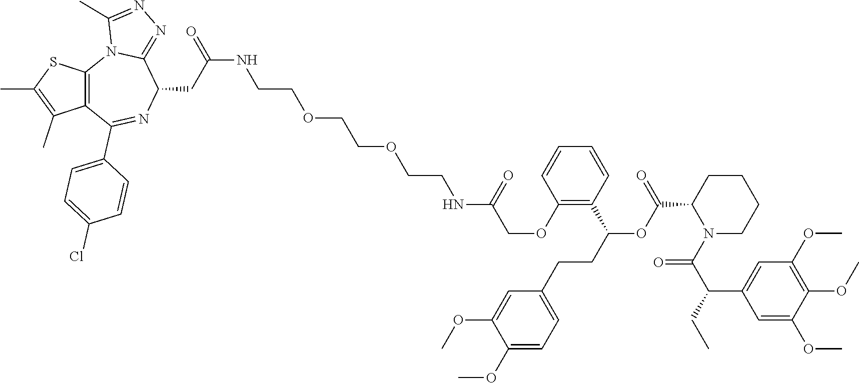

39. A composition comprising the structure of

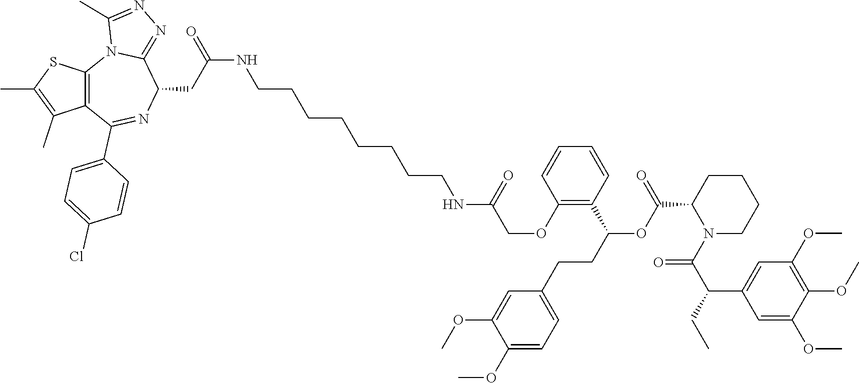

40. A composition comprising the structure of

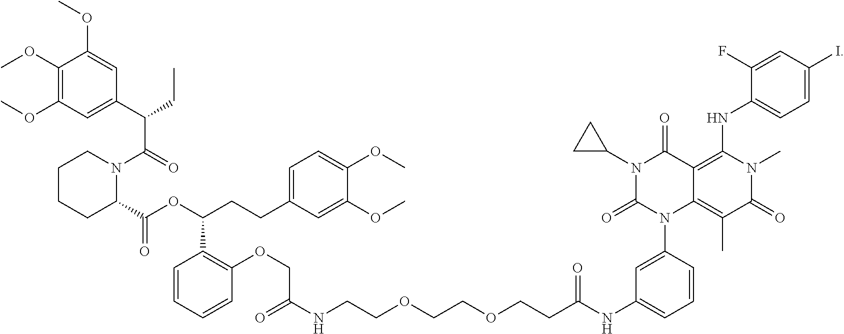 [SMILES: CC[C@H](C(=O)N1CCCC[C@H]1C(=O)O[C@H](CCc1ccc(OC)c(OC)c1)c1ccccc1OCC(=O)NCCOCCOCCC(=O)Nc1cccc(-n2c(=O)n(C3CC3)c(=O)c3c(Nc4ccc(I)cc4F)n(C)c(=O)c(C)c32)c1)c1cc(OC)c(OC)c(OC)c1]

41. A system comprising a labeling composition and a targeting composition,
wherein the labeling composition comprises a ligandable tag, a linker, and a labeling enzyme, wherein the linker attaches the ligandable tag to the labeling enzyme,
wherein the targeting composition comprises a ligand conjugated to a molecule of interest,
wherein the ligand of the targeting composition is a ligand for the ligandable tag of the labeling composition.

42. The system of claim 41, wherein the labeling composition and the targeting composition are bound to each other.

43. A method of detecting a binding partner of a molecule of interest comprising
a) adding a labeling composition and a target composition to a sample,
wherein the labeling composition comprises a ligandable tag conjugated to a labeling enzyme,
wherein the target composition comprises a ligand and a molecule of interest,
wherein the ligand of the target composition is a ligand for the ligandable tag of the labeling composition;
b) incubating the labeling composition and target composition with the sample under conditions that allow the molecule of interest to bind to a binding partner in the sample, wherein when the molecule of interest binds to a binding partner in the sample, the labeling enzyme labels the binding partner,
c) detecting the presence of the label in the sample,
wherein the presence of a label is indicative of a binding partner of a molecule of interest present in the sample.

44. The method of claim 43, wherein the labeling composition is added to a cell of the sample using transfection, transduction, or genetic engineering.

45. The method of any one of claims 43-44, wherein the labeling enzyme is any enzyme which can label a protein for biochemical analysis.

46. The method of any one of claims 43-45, wherein the labeling enzyme is a protein ligase.

47. The method of any one of claims 43-46, wherein the protein ligase is a biotin ligase.

48. The method of claim 47, wherein the biotin ligase is miniTurboID, TurboID, or BirA.

49. The method of any one of claims 47-48, wherein the protein ligase is Formlyglycine, sortase, transglutaminase, famesyltransferase, or lipoic acid ligase.

50. The method of any one of claims 43-49, wherein the ligandable tag is FKBP^{F36V}.

51. The method of any one of claims 43-50, wherein the ligandable tag is Halo TAG, SNAp-tag, CLIp-TAG, LigandLink, or cutinase.

52. The method of any one of claims 43-51, wherein the composition comprises a linker between the ligand and molecule of interest.

53. The method of claim 52, wherein the linker is a chemical linker.

54. A method of identifying a binding partner of a molecule of interest comprising
a) adding a labeling composition and a targeting composition to a sample,
wherein the labeling composition comprises a ligandable tag, a linker, and a labeling enzyme, wherein the linker attaches the ligandable tag to the labeling enzyme,
wherein the targeting composition comprises a ligand conjugated to a molecule of interest,
wherein the ligand of the targeting composition is a ligand for the ligandable tag of the labeling composition;
b) incubating the labeling composition and targeting composition with the sample under conditions that allow the molecule of interest to bind to a binding partner in the sample, wherein when the molecule of interest binds to a binding partner in the sample, wherein the labeling enzyme labels the binding partner;
c) detecting the presence of the label in the sample, and
d) identifying the labeled binding partner.

55. The method of claim 54, further comprising isolating the labeled binding partner from the molecule of interest prior to identifying.

56. The method of claim 54, wherein identifying the labeled binding partner comprises isolating any labeled molecules from the sample.

57. The method of any one of claims 54-56, wherein identifying the labeled binding partner comprises performing proteomics, immunoblotting, or imaging.

58. The method of any one of claims 54-57, wherein the labeling enzyme is a protein ligase.

59. The method of claim 58, wherein the protein ligase is any enzyme which can label a protein for biochemical analysis.

60. The method of any one of claims 54-59, wherein the protein ligase is a biotin ligase.

61. The method of claim 60, wherein the biotin ligase is miniTurboID, TurboID, or BirA.

62. The method of any one of claims 54-61, wherein the protein ligase is Formlyglycine, sortase, transglutaminase, famesyltransferase, APEX, or lipoic acid ligase.

63. The method of any one of claims 54-62, wherein the ligandable tag is FKBP12^{F36V}.

64. The method of any one of claims 54-63, wherein the ligandable tag is Halo TAG, SNAp-tag, CLIp-TAG, LigandLink, or cutinase.

65. The method of any one of claims 54-64, wherein the composition comprises a linker between the ligand and molecule of interest.

66. The method of claim 65, wherein the linker is a chemical linker.

67. The method of any one of claims 54-66, wherein the ligand is Ortho-AP1867.


SEQUENCE LISTING (4 sequences)

SEQ ID NO: 1 - protein, 422 aa, synthetic construct
MARDPPVATMIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEKASGGSGGGSGGGSGGGSGGGSGGGSGGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGYPYDVPDYAGYPYDVPDYAG

SEQ ID NO: 2 - protein, 24 aa, synthetic construct
GGSGGGSGGGSGGGSGGGSGGGSG

SEQ ID NO: 3 - DNA, 6662 bp, synthetic construct
  source 1..6662: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggctcgggatccaccggtcgccaccatgatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaaggctagcggtggttctggtggcggcagtgggggggggtccggtggcggatcaggtggtggaagtggcggtggctcaggtggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggcggctacccctacgacgtgcccgactacgccggctatccgtatgatgtcccggactatgcaggctaagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 1269 bp, synthetic construct
  source 1..1269: mol_type = other DNA
atggctcgggatccaccggtcgccaccatgatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaaggctagcggtggttctggtggcggcagtgggggggggtccggtggcggatcaggtggtggaagtggcggtggctcaggtggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggcggctacccctacgacgtgcccgactacgccggctatccgtatgatgtcccggactatgcaggctaa